(19) United States
(12) Patent Application Publication
(10) Pub. No.: US 2023/0047732 A1
(43) Pub. Date: Feb. 16, 2023

(54) PYRAZOLYLPYRIMIDINES AND USE THEREOF

(71) Applicant: BioTheryX, Inc., San Diego, CA (US)

(72) Inventors: Kyle W.H. Chan, San Diego, CA (US); Robert W. Sullivan, Vista, CA (US)

(21) Appl. No.: 17/758,693

(22) PCT Filed: Jan. 12, 2021
(86) PCT No.: PCT/US2021/013138

§ 371 Date: Jul. 12, 2022

Related U.S. Application Data
(60) Provisional application No. 62/960,646, filed on Jan. 13, 2020.

(51) Int. Cl. A61K 31/496; A61K 9/20; A61K 9/00

(52) U.S. Cl. CPC A61K 31/496; A61K 9/20; A61K 9/0056

(57) ABSTRACT

Provided herein is a method of treating, preventing, or ameliorating one or more symptoms of relapsed or refractory acute myeloid leukemia or high-risk myelodysplastic syndrome using a pyrazolylpyrimidine, e.g., a compound of Formula (I).

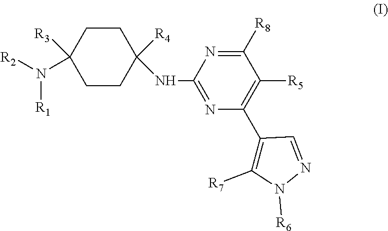

### CROSS REFERENCE TO RELATED APPLICATION

This application claims the benefit of the priority of U.S. Provisional Application No. 62/960,646, filed Jan. 13, 2020; the disclosure of which is incorporated herein by reference in its entirety.

### FIELD

Provided herein is a method of treating, preventing, or ameliorating one or more symptoms of relapsed or refractory acute myeloid leukemia or high-risk myelodysplastic syndrome using a pyrazolylpyrimidine.

### BACKGROUND

Casein kinases are serine/threonine kinases that phosphorylate proteins to mediate normal biological functions and malignant transformation. Schittek et al., Mol. Cancer 2014, 13, 231-245. Casein kinase 1 alpha (CK1α) functions as a tumor inducer in several cancers through negative regulation of Wnt/β-catenin signaling and p53. Ebert & Krönke, N. Engl. J. Med. 2018, 379, 1873-1874. CK1α phosphorylates β-catenin at serine 45, leading to ubiquitination and degradation of β-catenin. Schittek et al., Mol. Cancer 2014, 13, 231-245; Ebert & Krönke, N. Engl. J. Med. 2018, 379, 1873-1874; Elyada et al., Nature 2011, 470, 409-413. CK1α also phosphorylates murine double minute X (MDMX) at serine 289, resulting in enhanced binding of MDMX to p53. Wu et al., Mol. Cell. Biol. 2012, 32, 4821-4832. Additionally, a complex of CK1α and mouse double minute 2 homolog (MDM2) inhibits p53. Elyada et al., Nature 2011, 470, 409-413. Thus, enhanced inhibition of CKlu with subsequent p53 activation has the potential to be effective in treating a wide array of cancers.

CK1α is encoded by a deleted region in deletion 5q (del(5q)), a common cytogenetic abnormality in both acute myeloid leukemia (AML) and myelodysplastic syndromes (MDS). In mice, CK1α haploinsufficiency promotes aberrant hematopoietic stem cell self-renewal. Krönke et al., Nature 2015, 523, 183-188. At the same time, complete loss of CK1α results in complete failure of hematopoiesis. Schneider et al., Cancer Cell. 2014, 26, 509-520.

AML and MDS are heterogeneous clonal neoplasms of hematopoietic stem cells that are characterized by proliferation of abnormal immature blasts in the bone marrow (BM) and ineffective hematopoiesis leading to cytopenias. For those with relapsed or refractory AML, additional salvage chemotherapy produces remissions in only 20% to 25% of patients. Rosenblat et al., Clin. Cancer Res. 2010, 16, 5303-5311. Similarly, patients with MDS after progression on hypomethylating agents have particularly poor outcomes with a median survival of 4 to 6 months. Duong et al., Clin. Lymphoma Myeloma Leuk. 2013, 13, 711-715; Prebet et al., Br. J. Haematol. 2012, 157, 764 766; Jabbour et al., Cancer 2010, 116, 3830-3834. Thus, there is a need of an effective therapy for treating AML and MDS, in particular, relapsed or refractory AML or MDS.

### SUMMARY OF THE DISCLOSURE

Provided herein is a method of treating, preventing, or ameliorating one or more symptoms of relapsed or refractory acute myeloid leukemia (AML) or high-risk myelodysplastic syndrome (MDS), comprising administering to a subject in need thereof a therapeutically effective amount of a compound of Formula (I),

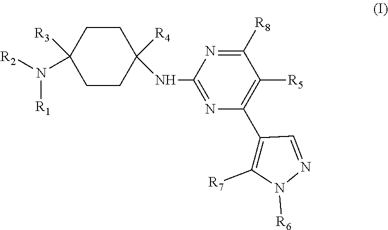

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:

R₁ and R₂ are each independently (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; or

R₁ and R₂ together with the nitrogen atom to which they are attached form heteroaryl or heterocyclyl;

R₃ and R₄ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; or

R₁ or R₂ together with R₃ and the carbon and nitrogen atom to which they are attached form heterocyclyl;

R₅, R₇, and R₈ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};

R₆ is hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

wherein each alkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};

wherein each Q^{a} is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Also provided herein is (1r,4r)-N¹-(5-chloro-4-(5-(cyclopropylmethyl)-1H-pyrazol-4-yl)pyrimidin-2-yl)cyclohexane-1,4-diamine, or a tautomer, a mixture of two or more tautomers, or an isotopic variant thereof; or a pharmaceutically acceptable salt, solvate, hydrate, or prodrug thereof.

Furthermore, provided herein is a method of inhibiting the growth of a cell, comprising contacting the cell with an effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Additionally, provided herein is a method of modulating the activity of CK1α in a cell, comprising contacting the cell with an effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of inducing apoptosis in a cell, comprising contacting the cell with an effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human or an animal) without excessive toxicity, irritation, allergic response, immunogenicity, or other problems or complications, commensurate with a reasonable benefit/risk ratio. See, e.g., Remington: The Science and Practice of Pharmacy, 22nd ed.; Allen Ed.: Philadelphia, Pa., 2012; Handbook of Pharmaceutical Excipients, 8th ed.; Sheskey et al., Eds.; The Pharmaceutical Press: 2017; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, sec-butyl, t-butyl, pentyl (including all isomeric forms), and hexyl (including all isomeric forms).

The term “alkenyl” refers to a linear or branched monovalent hydrocarbon radical, which contains one or more, in one embodiment, one, two, three, four, or five, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl.

The term “alkynyl” refers to a linear or branched monovalent hydrocarbon radical, which contains one or more, in one embodiment, one, two, three, four, or five, in another embodiment, one, carbon-carbon triple bond(s). The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl).

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is polycyclic. In yet another embodiment, the aryl is bicyclic. In still another embodiment, the aryl is tricyclic. In certain embodiments, the aryl is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, 2-phenylethyl, and 3-phenylpropyl. In certain embodiments, the aralkyl is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. The heteroaryl is bonded to the rest of a molecule through the aromatic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. In yet another embodiment, the heteroaryl is tricyclic. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, the heteroaryl is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. The heterocyclyl is bonded to the rest of a molecule through the non-aromatic ring. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, the heterocyclyl is optionally substituted with one or more substituents Q as described herein.

The term “halogen”, “halide,” or “halo” refers to fluorine, chlorine, bromine, and/or iodine.

The term “optionally substituted” is intended to mean that a group or substituent, such as an alkyl, alkenyl, alkynyl, cycloalkyl, aryl, aralkyl, heteroaryl, or heterocyclyl group, may be substituted with one or more, one, two, three, or four, substituents Q, each of which is independently selected from, e.g., (a) deuterium (-D), cyano (—CN), halo, and nitro (—NO₂); (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(═NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(═NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{e}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{e} together with the N atom to which they are attached form heterocyclyl, optionally substituted with one or more, in one embodiment, one, two, three, or four, substituents Q^{a}. As used herein, all groups that can be substituted are “optionally substituted,” unless otherwise specified.

In one embodiment, each Q^{a} is independently selected from the group consisting of (a) deuterium, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(═NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(═NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{c}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O) oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (2H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I) It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (2H or D), and tritium (3H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean sufficiently homogeneous to appear free of readily detectable impurities as determined by standard analytical methods used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof” has the same meaning as the phrase “(i) an enantiomer, a mixture of enantiomers, a diastereomer, a mixture of two or more diastereomers, a tautomer, a mixture of two or more tautomers, or an isotopic variant of the compound referenced therein; or (ii) a pharmaceutically acceptable salt, solvate, hydrate, or prodrug of the compound referenced therein, or (iii) a pharmaceutically acceptable salt, solvate, hydrate, or prodrug of an enantiomer, a mixture of enantiomers, a diastereomer, a mixture of two or more diastereomers, a tautomer, a mixture of two or more tautomers, or an isotopic variant of the compound referenced therein.”

### Pyrazolylpyrimidine Compounds

In one embodiment, described herein is a compound of Formula (I),

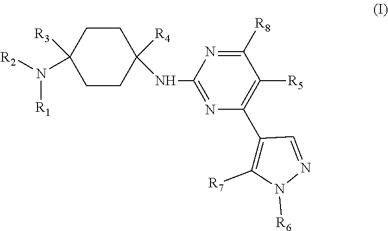

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein:

R₁ and R₂ are each independently (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; or

R₁ and R₂ together with the nitrogen atom to which they are attached form heteroaryl or heterocyclyl;

R₃ and R₄ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; or

R₁ or R₂ together with R₃ and the carbon and nitrogen atom to which they are attached form heterocyclyl;

R₅, R₇, and R₈ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};

R₆ is hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and

each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

wherein each alkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};

wherein each Q^{a} is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR C(O)NR^{f}R^{g}, —NR^{e}C(O)SR^{f}, —NR C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, R₁ and R₂ are each independently hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl. In certain embodiments, R₁ and R₂ are each independently —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}.

In certain embodiments, R₁ is hydrogen. In certain embodiments, R₂ is hydrogen. In certain embodiments, R₁ and R₂ are each hydrogen.

In certain embodiments, R₃ is hydrogen. In certain embodiments, R₄ is hydrogen. In certain embodiments, R₃ and R₄ are each hydrogen.

In certain embodiments, R₅ is hydrogen, deuterium, cyano, halo, nitro, or C₁₋₆ alkyl. In certain embodiments, R₅ is halo. In certain embodiments, R₅ is Cl.

In certain embodiments, R₆ is hydrogen or C₁₋₆ alkyl optionally substituted with one or more substituents Q. In certain embodiments, R₆ is hydrogen. In certain embodiments, R₆ is methyl.

In certain embodiments, R₇ is C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; each of which is optionally substituents with one or more substituents Q. In certain embodiments, R₇ is C₁₋₆ alkyl optionally substituents with one or more substituents Q. In certain embodiments, R₇ is C₁₋₆ alkyl substituted with C₃₋₁₀ cycloalkyl, i.e., C₃₋₁₀ cycloalkyl-C₁₋₆ alkyl, wherein the alkyl and cycloalkyl are each optionally substituents with one or more substituents Q^{a}. In certain embodiments, R₇ is cyclopropylmethyl, optionally substituents with one or more substituents Q.

In certain embodiments, R₇ is —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

In certain embodiments, R₈ is (i) hydrogen, deuterium, cyano, halo, or nitro; or (ii) C₁₋₆ alkyl optionally substituted with one or more substituents Q. In certain embodiments, R₈ is hydrogen.

In another embodiment, described herein is:

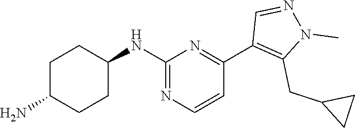 [SMILES: Cn1ncc(-c2ccnc(N[C@H]3CC[C@H](N)CC3)n2)c1CC1CC1]

(1r,4r)-N¹-(4-(5-(cyclopropylmethyl)-1-methyl-1H-pyrazol-4-yl)pyrimidin-2-yl)cyclohexane-1,4-diamine A1;

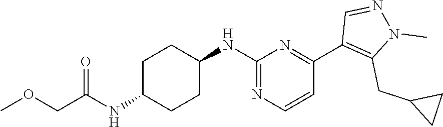 [SMILES: COCC(=O)N[C@H]1CC[C@H](Nc2nccc(-c3cnn(C)c3CC3CC3)n2)CC1]

N-((1r,4r)-4-((4-(5-(cyclopropylmethyl)-1-methyl-1H-pyrazol-4-yl)pyrimidin-2-yl)amino)cyclohexyl)-2-methoxyacetamide A2;

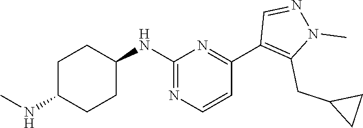 [SMILES: CN[C@H]1CC[C@H](Nc2nccc(-c3cnn(C)c3CC3CC3)n2)CC1]

(1r,4r)-N¹-(4-(5-(cyclopropylmethyl)-1-methyl-1H-pyrazol-4-yl)pyrimidin-2-yl)-N⁴-methylcyclohexane-1,4-diamine A3;

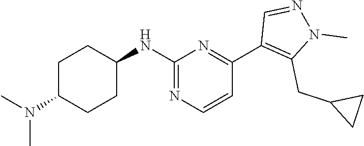 [SMILES: Cn1ncc(-c2ccnc(N[C@H]3CC[C@H](N(C)C)CC3)n2)c1CC1CC1]

(1r,4r)-N¹-(4-(5-(cyclopropylmethyl)-1-methyl-1H-pyrazol-4-yl)pyrimidin-2-yl)-N⁴,N⁴-dimethylcyclohexane-1,4-diamine A4;

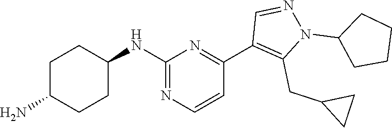 [SMILES: N[C@H]1CC[C@H](Nc2nccc(-c3cnn(C4CCCC4)c3CC3CC3)n2)CC1]

(1r,4r)-N¹-(4-(1-cyclopentyl-5-(cyclopropylmethyl)-1H-pyrazol-4-yl)pyrimidin-2-yl)cyclohexane-1,4-diamine A5;

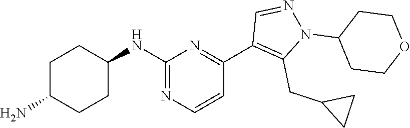 [SMILES: N[C@H]1CC[C@H](Nc2nccc(-c3cnn(C4CCOCC4)c3CC3CC3)n2)CC1]

(1r,4r)-N¹-(4-(5-(cyclopropylmethyl)-1-(tetrahydro-2H-pyran-4-yl)-1H-pyrazol-4-yl)pyrimidin-2-yl)cyclohexane-1,4-diamine A6;

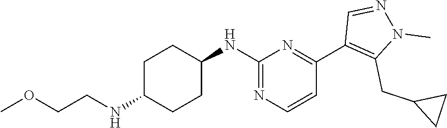 [SMILES: COCCN[C@H]1CC[C@H](Nc2nccc(-c3cnn(C)c3CC3CC3)n2)CC1]

(1r,4r)-N¹-4-(5-(cyclopropylmethyl)-1-methyl-1H-pyrazol-4-yl)pyrimidin-2-yl)-N⁴-(2-methoxyethyl)cyclohexane-1,4-diamine A7;

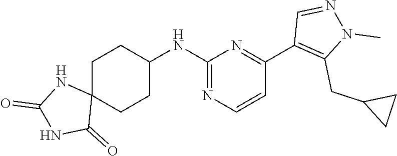 [SMILES: Cn1ncc(-c2ccnc(NC3CCC4(CC3)NC(=O)NC4=O)n2)c1CC1CC1]

8-((4-(5-(cyclopropylmethyl)-1-methyl-1H-pyrazol-4-yl)pyrimidin-2-yl)amino)-1,3-diazaspiro[4.5]decane-2,4-dione A8;

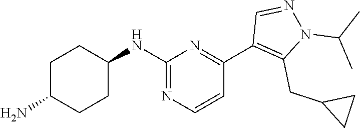 [SMILES: CC(C)n1ncc(-c2ccnc(N[C@H]3CC[C@H](N)CC3)n2)c1CC1CC1]

(1r,4r)-N¹-(4-(5-(cyclopropylmethyl)-1-isopropyl-1H-pyrazol-4-yl)pyrimidin-2-yl)cyclohexane-1,4-diamine A9;

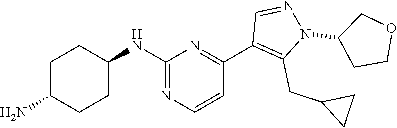

(1r,4S)—N¹-(4-(5-(cyclopropylmethyl)-1-((S)-tetrahydrofuran-3-yl)-1H-pyrazol-4-yl)pyrimidin-2-yl)cyclohexane-1,4-diamine A10;

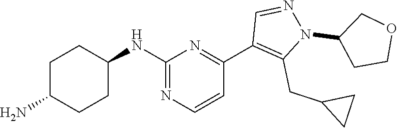

(1r,4S)—N¹-(4-(5-(cyclopropylmethyl)-1-((S)-tetrahydrofuran-3-yl)-1H-pyrazol-4-yl)pyrimidin-2-yl)cyclohexane-1,4-diamine A11;

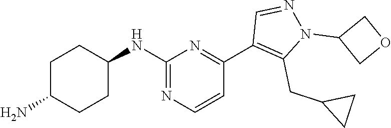 [SMILES: N[C@H]1CC[C@H](Nc2nccc(-c3cnn(C4COC4)c3CC3CC3)n2)CC1]

(1r,4r)-N¹-(4-(5-(cyclopropylmethyl)-1-(oxetan-3-yl)-1H-pyrazol-4-yl)pyrimidin-2-yl)cyclohexane-1,4-diamine A12;

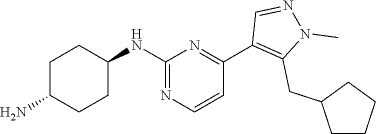 [SMILES: Cn1ncc(-c2ccnc(N[C@H]3CC[C@H](N)CC3)n2)c1CC1CCCC1]

(1r,4r)-N¹-(4-(5-(cyclopentylmethyl)-1-methyl-1H-pyrazol-4-yl)pyrimidin-2-yl)cyclohexane-1,4-diamine A13;

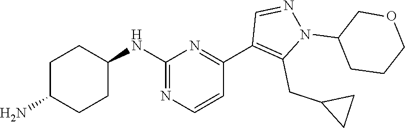 [SMILES: N[C@H]1CC[C@H](Nc2nccc(-c3cnn(C4CCCOC4)c3CC3CC3)n2)CC1]

(1r,4r)-N¹-(4-(5-(cyclopropylmethyl)-1-(tetrahydro-2H-pyran-3-yl)-1H-pyrazol-4-yl)pyrimidin-2-yl)cyclohexane-1,4-diamine A14;

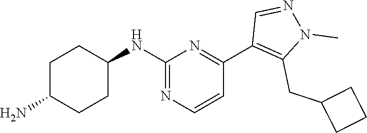 [SMILES: Cn1ncc(-c2ccnc(N[C@H]3CC[C@H](N)CC3)n2)c1CC1CCC1]

(1r,4r)-N¹-4-(5-(cyclobutylmethyl)-1-methyl-1H-pyrazol-4-yl)pyrimidin-2-yl)cyclohexane-1,4-diamine A15;

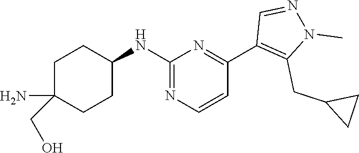

(1-amino-4-((4-(5-(cyclopropylmethyl)-1-methyl-1H-pyrazol-4-yl)pyrimidin-2-yl)amino)cyclohexyl)methanol A16;

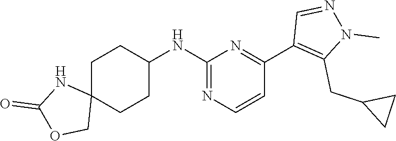 [SMILES: Cn1ncc(-c2ccnc(NC3CCC4(CC3)COC(=O)N4)n2)c1CC1CC1]

8-((4-(5-(cyclopropylmethyl)-1-methyl-1H-pyrazol-4-yl)pyrimidin-2-yl)amino)-3-oxa-1-azaspiro[4.5]decan-2-one A17;

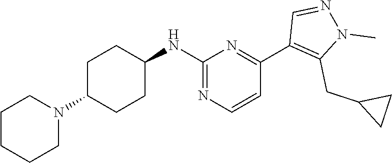 [SMILES: Cn1ncc(-c2ccnc(N[C@H]3CC[C@H](N4CCCCC4)CC3)n2)c1CC1CC1]

4-(5-(cyclopropylmethyl)-1-methyl-1H-pyrazol-4-yl)-N-((1r,4r)-4-(piperidin-1-yl)cyclohexyl)pyrimidin-2-amine A18;

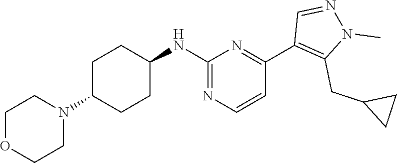 [SMILES: Cn1ncc(-c2ccnc(N[C@H]3CC[C@H](N4CCOCC4)CC3)n2)c1CC1CC1]

4-(5-(cyclopropylmethyl)-1-methyl-1H-pyrazol-4-yl)-N-((1r,4r)-4-morpholinocyclohexyl)-pyrimidin-2-amine A19;

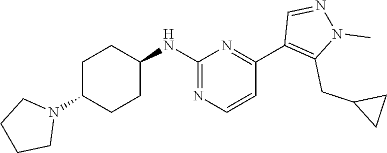 [SMILES: Cn1ncc(-c2ccnc(N[C@H]3CC[C@H](N4CCCC4)CC3)n2)c1CC1CC1]

4-(5-(cyclopropylmethyl)-1-methyl-1H-pyrazol-4-yl)-N-((1r,4r)-4-(pyrrolidin-1-yl)cyclohexyl)pyrimidin-2-amine A20;

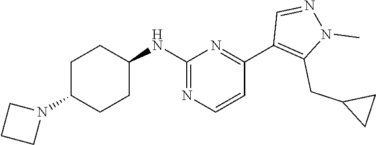 [SMILES: Cn1ncc(-c2ccnc(N[C@H]3CC[C@H](N4CCC4)CC3)n2)c1CC1CC1]

N-((1r,4r)-4-(azetidin-1-yl)cyclohexyl)-4-(5-(cyclopropylmethyl)-1-methyl-1H-pyrazol-4-yl)pyrimidin-2-amine A21;

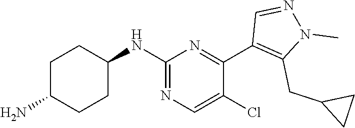 [SMILES: Cn1ncc(-c2nc(N[C@H]3CC[C@H](N)CC3)ncc2Cl)c1CC1CC1]

(1r,4r)-N¹-(5-chloro-4-(5-(cyclopropylmethyl)-1-methyl-1H-pyrazol-4-yl)pyrimidin-2-yl)cyclohexane-1,4-diamine A22;

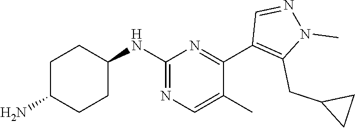 [SMILES: Cc1cnc(N[C@H]2CC[C@H](N)CC2)nc1-c1cnn(C)c1CC1CC1]

(1r,4r)-N¹-(4-(5-(cyclopropylmethyl)-1-methyl-1H-pyrazol-4-yl)-5-methylpyrimidin-2-yl)cyclohexane-1,4-diamine A23;

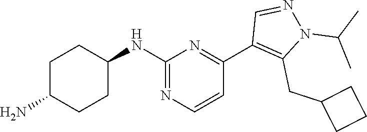 [SMILES: CC(C)n1ncc(-c2ccnc(N[C@H]3CC[C@H](N)CC3)n2)c1CC1CCC1]

(1r,4r)-N¹-(4-(5-(cyclobutylmethyl)-1-isopropyl-1H-pyrazol-4-yl)pyrimidin-2-yl)cyclohexane-1,4-diamine A24;

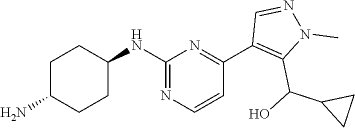 [SMILES: Cn1ncc(-c2ccnc(N[C@H]3CC[C@H](N)CC3)n2)c1C(O)C1CC1]

(4-(2-(((1r,4r)-4-aminocyclohexyl)amino)pyrimidin-4-yl)-1-methyl-1H-pyrazol-5-yl)(cyclopropyl)methanol A25;

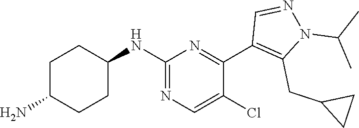 [SMILES: CC(C)n1ncc(-c2nc(N[C@H]3CC[C@H](N)CC3)ncc2Cl)c1CC1CC1]

(1r,4r)-N¹-(5-chloro-4-(5-(cyclopropylmethyl)-1-isopropyl-1H-pyrazol-4-yl)pyrimidin-2-yl)cyclohexane-1,4-diamine A26;

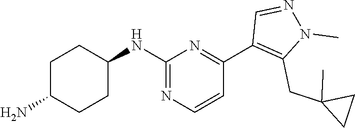 [SMILES: Cn1ncc(-c2ccnc(N[C@H]3CC[C@H](N)CC3)n2)c1CC1(C)CC1]

(1r,4r)-N¹-4-(1-methyl-5-((1-methylcyclopropyl)methyl)-1H-pyrazol-4-yl)pyrimidin-2-yl)cyclohexane-1,4-diamine A27;

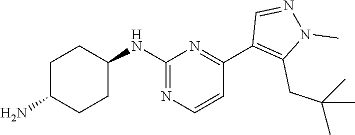 [SMILES: Cn1ncc(-c2ccnc(N[C@H]3CC[C@H](N)CC3)n2)c1CC(C)(C)C]

(1 r,4r)-N¹-4-(1-methyl-5-neopentyl-1H-pyrazol-4-yl)pyrimidin-2-yl)cyclohexane-1,4-diamine A28;

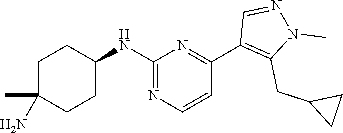 [SMILES: Cn1ncc(-c2ccnc(N[C@H]3CC[C@](C)(N)CC3)n2)c1CC1CC1]

(1r,4r)-N¹-(4-(5-(cyclopropylmethyl)-1-methyl-1H-pyrazol-4-yl)pyrimidin-2-yl)-4-methylcyclohexane-1,4-diamine A29;

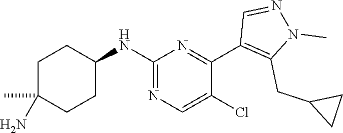 [SMILES: Cn1ncc(-c2nc(N[C@H]3CC[C@@](C)(N)CC3)ncc2Cl)c1CC1CC1]

is (1s,4s)-N¹-(5-chloro-4-(5-(cyclopropylmethyl)-1-methyl-1H-pyrazol-4-yl)pyrimidin-2-yl)-4-methylcyclohexane-1,4-diamine A30;

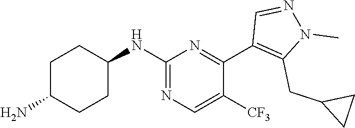 [SMILES: Cn1ncc(-c2nc(N[C@H]3CC[C@H](N)CC3)ncc2C(F)(F)F)c1CC1CC1]

(1r,4r)-N¹-(4-(5-(cyclopropylmethyl)-1-methyl-1H-pyrazol-4-yl)-5-(trifluoromethyl)-pyrimidin-2-yl)cyclohexane-1,4-diamine A31;

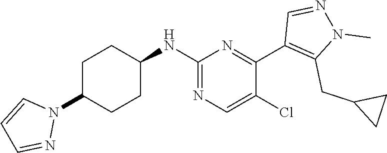 [SMILES: Cn1ncc(-c2nc(N[C@H]3CC[C@@H](n4cccn4)CC3)ncc2Cl)c1CC1CC1]

N-((1r,4r)-4-(1H-pyrazol-1-yl)cyclohexyl)-5-chloro-4-(5-(cyclopropylmethyl)-1-methyl-1H-pyrazol-4-yl)pyrimidin-2-amine A32;

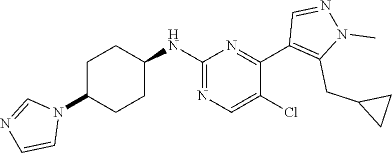 [SMILES: Cn1ncc(-c2nc(N[C@H]3CC[C@@H](n4ccnc4)CC3)ncc2Cl)c1CC1CC1]

N-((1r,4r)-4-(1H-imidazol-1-yl)cyclohexyl)-5-chloro-4-(5-(cyclopropylmethyl)-1-methyl-1H-pyrazol-4-yl)pyrimidin-2-amine A33;

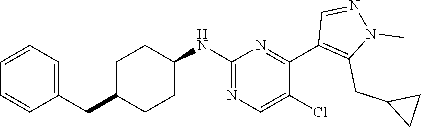 [SMILES: Cn1ncc(-c2nc(N[C@H]3CC[C@@H](Cc4ccccc4)CC3)ncc2Cl)c1CC1CC1]

(1r,4r)-N¹-(5-chloro-4-(5-(cyclopropylmethyl)-1-methyl-1H-pyrazol-4-yl)pyrimidin-2-yl)-N⁴-phenylcyclohexane-1,4-diamine A34;

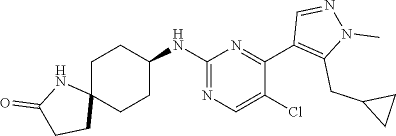

(5r,8r)-8-((5-chloro-4-(5-(cyclopropylmethyl)-1-methyl-1H-pyrazol-4-yl)pyrimidin-2-yl)amino)-1-azaspiro[4.5]decan-2-one A35;

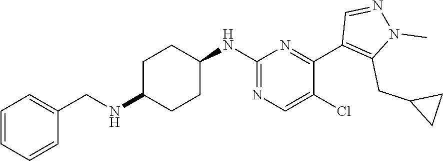 [SMILES: Cn1ncc(-c2nc(N[C@H]3CC[C@@H](NCc4ccccc4)CC3)ncc2Cl)c1CC1CC1]

(1r,4r)-N¹-benzyl-N⁴-(5-chloro-4-(5-(cyclopropylmethyl)-1-methyl-1H-pyrazol-4-yl)pyrimidin-2-yl)cyclohexane-1,4-diamine A36;

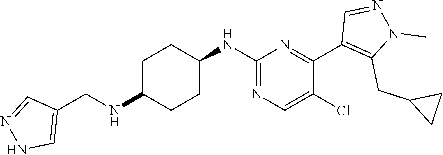 [SMILES: Cn1ncc(-c2nc(N[C@H]3CC[C@@H](NCc4cn[nH]c4)CC3)ncc2Cl)c1CC1CC1]

(1r,4r)-N¹-((1H-pyrazol-4-yl)methyl)-N⁴-(5-chloro-4-(5-(cyclopropylmethyl)-1-methyl-1H-pyrazol-4-yl)pyrimidin-2-yl)cyclohexane-1,4-diamine A37;

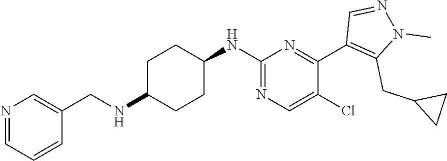 [SMILES: Cn1ncc(-c2nc(N[C@H]3CC[C@@H](NCc4cccnc4)CC3)ncc2Cl)c1CC1CC1]

(1r,4r)-N¹-(5-chloro-4-(5-(cyclopropylmethyl)-1-methyl-1H-pyrazol-4-yl)pyrimidin-2-yl)-N⁴-(pyridin-3-ylmethyl)cyclohexane-1,4-diamine A38;

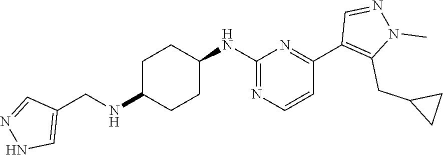 [SMILES: Cn1ncc(-c2ccnc(N[C@H]3CC[C@@H](NCc4cn[nH]c4)CC3)n2)c1CC1CC1]

(1r,4r)-N¹-((1H-pyrazol-4-yl)methyl)-N⁴-(4-(5-(cyclopropylmethyl)-1-methyl-1H-pyrazol-4-yl)pyrimidin-2-yl)cyclohexane-1,4-diamine A39;

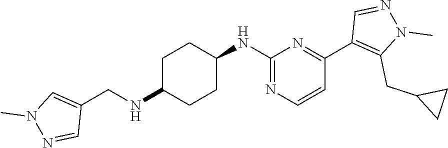 [SMILES: Cn1cc(CN[C@H]2CC[C@@H](Nc3nccc(-c4cnn(C)c4CC4CC4)n3)CC2)cn1]

(1r,4r)-N¹-(4-(5-(cyclopropylmethyl)-1-methyl-1H-pyrazol-4-yl)pyrimidin-2-yl)-N⁴-((1-methyl-1H-pyrazol-4-yl)methyl)cyclohexane-1,4-diamine A40;

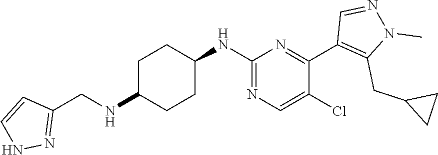 [SMILES: Cn1ncc(-c2nc(N[C@H]3CC[C@@H](NCc4cc[nH]n4)CC3)ncc2Cl)c1CC1CC1]

(1r,4r)-N¹-((1H-pyrazol-5-yl)methyl)-N⁴-(5-chloro-4-(5-(cyclopropylmethyl)-1-methyl-1H-pyrazol-4-yl)pyrimidin-2-yl)cyclohexane-1,4-diamine A41;

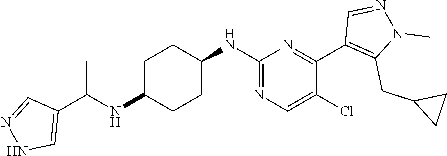 [SMILES: CC(N[C@H]1CC[C@@H](Nc2ncc(Cl)c(-c3cnn(C)c3CC3CC3)n2)CC1)c1cn[nH]c1]

(1r,4r)-N¹-(1-(1H-pyrazol-4-yl)ethyl)-N⁴-(5-chloro-4-(5-(cyclopropylmethyl)-1-methyl-1H-pyrazol-4-yl)pyrimidin-2-yl)cyclohexane-1,4-diamine A42;

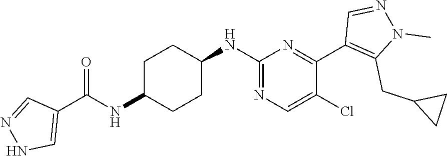 [SMILES: Cn1ncc(-c2nc(N[C@H]3CC[C@@H](NC(=O)c4cn[nH]c4)CC3)ncc2Cl)c1CC1CC1]

(1r,4r)-N¹-(5-chloro-4-(5-(cyclopropylmethyl)-1-methyl-1H-pyrazol-4-yl)pyrimidin-2-yl)-N⁴-phenylcyclohexane-1,4-diamine A43;

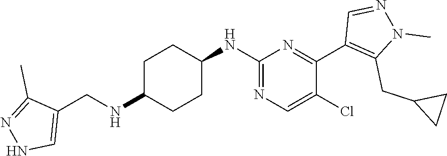 [SMILES: Cc1n[nH]cc1CN[C@H]1CC[C@@H](Nc2ncc(Cl)c(-c3cnn(C)c3CC3CC3)n2)CC1]

(1r,4r)-N¹-(5-chloro-4-(5-(cyclopropylmethyl)-1-methyl-1H-pyrazol-4-yl)pyrimidin-2-yl)-N⁴-((5-methyl-1H-pyrazol-4-yl)methyl)cyclohexane-1,4-diamine A44;

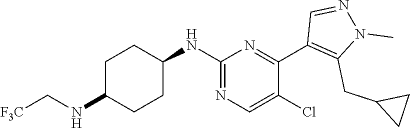 [SMILES: Cn1ncc(-c2nc(N[C@H]3CC[C@@H](NCC(F)(F)F)CC3)ncc2Cl)c1CC1CC1]

(1r,4r)-N¹-(5-chloro-4-(5-(cyclopropylmethyl)-1-methyl-1H-pyrazol-4-yl)pyrimidin-2-yl)-N⁴-(2,2,2-trifluoroethyl)cyclohexane-1,4-diamine A45;

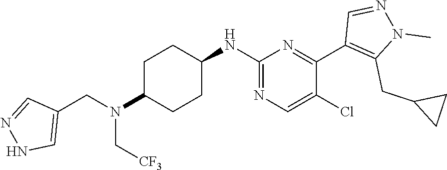 [SMILES: Cn1ncc(-c2nc(N[C@H]3CC[C@@H](N(Cc4cn[nH]c4)CC(F)(F)F)CC3)ncc2Cl)c1CC1CC1]

(1r,4r)-N¹-((1H-pyrazol-4-yl)methyl)-N⁴-(5-chloro-4-(5-(cyclopropylmethyl)-1-methyl-1H-pyrazol-4-yl)pyrimidin-2-yl)-N¹-(2,2,2-trifluoroethyl)cyclohexane-1,4-diamine A46;

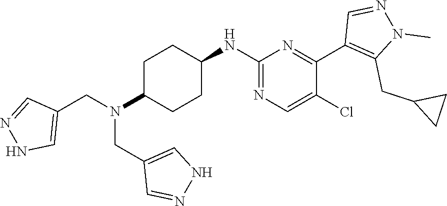 [SMILES: Cn1ncc(-c2nc(N[C@H]3CC[C@@H](N(Cc4cn[nH]c4)Cc4cn[nH]c4)CC3)ncc2Cl)c1CC1CC1]

(1r,4r)-N′,N′-bis((1H-pyrazol-4-yl)methyl)-N⁴-(5-chloro-4-(5-(cyclopropylmethyl)-1-methyl-1H-pyrazol-4-yl)pyrimidin-2-yl)cyclohexane-1,4-diamine A47;

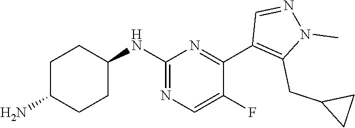 [SMILES: Cn1ncc(-c2nc(N[C@H]3CC[C@H](N)CC3)ncc2F)c1CC1CC1]

(1r,4r)-N′-(4-(5-(cyclopropylmethyl)-1-methyl-1H-pyrazol-4-yl)-5-fluoropyrimidin-2-yl)cyclohexane-1,4-diamine A48;

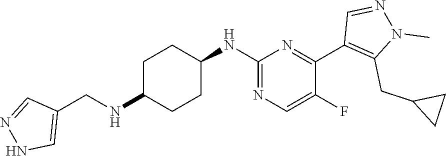 [SMILES: Cn1ncc(-c2nc(N[C@H]3CC[C@@H](NCc4cn[nH]c4)CC3)ncc2F)c1CC1CC1]

(1r,4r)-N¹-((1H-pyrazol-4-yl)methyl)-N⁴-(4-(5-(cyclopropylmethyl)-1-methyl-1H-pyrazol-4-yl)-5-fluoropyrimidin-2-yl)cyclohexane-1,4-diamine A49; or

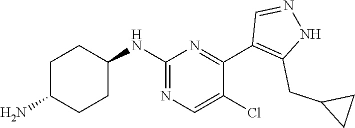 [SMILES: N[C@H]1CC[C@H](Nc2ncc(Cl)c(-c3cn[nH]c3CC3CC3)n2)CC1]

(1r,4r)-N¹-(5-chloro-4-(5-(cyclopropylmethyl)-1H-pyrazol-4-yl)pyrimidin-2-yl)cyclohexane-1,4-diamine A50;
   or a tautomer, a mixture of two or more tautomers, or an isotopic variant thereof; or a pharmaceutically acceptable salt, solvate, hydrate, or prodrug thereof.

In yet another embodiment, described herein is (1r,4r)-N¹-(5-chloro-4-(5-(cyclopropylmethyl)-1-methyl-1H-pyrazol-4-yl)pyrimidin-2-yl)cyclohexane-1,4-diamine A22, or a tautomer, a mixture of two or more tautomers, or an isotopic variant thereof; or a pharmaceutically acceptable salt, solvate, hydrate, or prodrug thereof.

In yet another embodiment, described herein is a p-toluensulfonate of (1r,4r)-N¹-(5-chloro-4-(5-(cyclopropylmethyl)-1-methyl-1H-pyrazol-4-yl)pyrimidin-2-yl)cyclohexane-1,4-diamine A22, or a tautomer, a mixture of two or more tautomers, or an isotopic variant thereof; or a pharmaceutically solvate or hydrate.

In yet another embodiment, described herein is a di-p-toluensulfonate of (1r,4r)-N¹-(5-chloro-4-(5-(cyclopropylmethyl)-1-methyl-1H-pyrazol-4-yl)pyrimidin-2-yl)cyclohexane-1,4-diamine A22, or a tautomer, a mixture of two or more tautomers, or an isotopic variant thereof; or a pharmaceutically solvate or hydrate.

In still another embodiment, described herein is (1r,4r)-N-(5-chloro-4-(5-(cyclopropylmethyl)-1H-pyrazol-4-yl)pyrimidin-2-yl)cyclohexane-1,4-diamine A50, or a tautomer, a mixture of two or more tautomers, or an isotopic variant thereof; or a pharmaceutically acceptable salt, solvate, hydrate, or prodrug thereof.

In certain embodiments, a compound described herein is deuterium-enriched. In certain embodiments, a compound described herein is carbon-13 enriched. In certain embodiments, a compound described herein is carbon-14 enriched. In certain embodiments, a compound described herein contains one or more less prevalent isotopes for other elements, including, but not limited to, ¹⁵N for nitrogen; ¹⁷O or ¹⁸O for oxygen, and ³³S, ³⁴S, or ³⁶S for sulfur.

In certain embodiments, a compound described herein has an isotopic enrichment factor of no less than about 5, no less than about 10, no less than about 20, no less than about 30, no less than about 40, no less than about 50, no less than about 60, no less than about 70, no less than about 80, no less than about 90, no less than about 100, no less than about 200, no less than about 500, no less than about 1,000, no less than about 2,000, no less than about 5,000, or no less than about 10,000. In any events, however, an isotopic enrichment factor for a specified isotope is no greater than the maximum isotopic enrichment factor for the specified isotope, which is the isotopic enrichment factor when a compound at a given position is 100% enriched with the specified isotope. Thus, the maximum isotopic enrichment factor is different for different isotopes. The maximum isotopic enrichment factor is 6410 for deuterium and 90 for carbon-13.

In certain embodiments, a compound described herein has a deuterium enrichment factor of no less than about 64 (about 1% deuterium enrichment), no less than about 130 (about 2% deuterium enrichment), no less than about 320 (about 5% deuterium enrichment), no less than about 640 (about 10% deuterium enrichment), no less than about 1,300 (about 20% deuterium enrichment), no less than about 3,200 (about 50% deuterium enrichment), no less than about 4,800 (about 75% deuterium enrichment), no less than about 5,130 (about 80% deuterium enrichment), no less than about 5,450 (about 85% deuterium enrichment), no less than about 5,770 (about 90% deuterium enrichment), no less than about 6,090 (about 95% deuterium enrichment), no less than about 6,220 (about 97% deuterium enrichment), no less than about 6,280 (about 98% deuterium enrichment), no less than about 6,350 (about 99% deuterium enrichment), or no less than about 6,380 (about 99.5% deuterium enrichment). The deuterium enrichment can be determined using conventional analytical methods known to one of ordinary skill in the art, including mass spectrometry and nuclear magnetic resonance spectroscopy.

In certain embodiments, a compound described herein has a carbon-13 enrichment factor of no less than about 1.8 (about 2% carbon-13 enrichment), no less than about 4.5 (about 5% carbon-13 enrichment), no less than about 9 (about 10% carbon-13 enrichment), no less than about 18 (about 20% carbon-13 enrichment), no less than about 45 (about 50% carbon-13 enrichment), no less than about 68 (about 75% carbon-13 enrichment), no less than about 72 (about 80% carbon-13 enrichment), no less than about 77 (about 85% carbon-13 enrichment), no less than about 81 (about 90% carbon-13 enrichment), no less than about 86 (about 95% carbon-13 enrichment), no less than about 87 (about 97% carbon-13 enrichment), no less than about 88 (about 98% carbon-13 enrichment), no less than about 89 (about 99% carbon-13 enrichment), or no less than about 90 (about 99.5% carbon-13 enrichment). The carbon-13 enrichment can be determined using conventional analytical methods known to one of ordinary skill in the art, including mass spectrometry and nuclear magnetic resonance spectroscopy.

In certain embodiments, at least one of the atoms of a compound described herein, as specified as isotopically enriched, has isotopic enrichment of no less than about 1%, no less than about 2%, no less than about 5%, no less than about 10%, no less than about 20%, no less than about 50%, no less than about 70%, no less than about 80%, no less than about 90%, or no less than about 98%. In certain embodiments, the atoms of a compound described herein, as specified as isotopically enriched, have isotopic enrichment of no less than about 1%, no less than about 2%, no less than about 5%, no less than about 10%, no less than about 20%, no less than about 50%, no less than about 70%, no less than about 80%, no less than about 90%, or no less than about 98%. In any events, the isotopic enrichment of the isotopically enriched atom of a compound described herein is no less than the natural abundance of the isotope specified.

In certain embodiments, at least one of the atoms of a compound described herein, as specified as deuterium-enriched, has deuterium enrichment of no less than about 1%, no less than about 2%, no less than about 5%, no less than about 10%, no less than about 20%, no less than about 50%, no less than about 70%, no less than about 80%, no less than about 90%, or no less than about 98%. In certain embodiments, the atoms of a compound described herein, as specified as deuterium-enriched, have deuterium enrichment of no less than about 1%, no less than about 2%, no less than about 5%, no less than about 10%, no less than about 20%, no less than about 50%, no less than about 70%, no less than about 80%, no less than about 90%, or no less than about 98%.

In certain embodiments, at least one of the atoms of a compound described herein, as specified as ¹³C-enriched, has carbon-13 enrichment of no less than about 2%, no less than about 5%, no less than about 10%, no less than about 20%, no less than about 50%, no less than about 70%, no less than about 80%, no less than about 90%, or no less than about 98%. In certain embodiments, the atoms of a compound described herein, as specified as ¹³C-enriched, have carbon-13 enrichment of no less than about 1%, no less than about 2%, no less than about 5%, no less than about 10%, no less than about 20%, no less than about 50%, no less than about 70%, no less than about 80%, no less than about 90%, or no less than about 98%.

In certain embodiments, a compound described herein is isolated or purified. In certain embodiments, a compound described herein has a purity of at least about 50%, at least about 70%, at least about 80%, at least about 90%, at least about 95%, at least about 98%, at least about 99%, or at least about 99.5% by weight.

The compounds described herein are intended to encompass all possible stereoisomers unless a particular stereochemistry is specified. Where a compound described herein contains an alkenyl group, the compound may exist as one or mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contain an aromatic moiety. It follows that a single compound may exhibit more than one type of isomerism.

A compound described herein can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When a compound described herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; Wiley-VCH and VHCA, Zurich, 2011. In certain embodiments, a pharmaceutically acceptable salt of a compound described herein is a hydrate.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid. In certain embodiments, a compound described herein is a hydrochloride salt. In certain embodiments, a compound described herein is a p-toluenesulfonate salt. In certain embodiments, a compound described herein is a di-p-toluenesulfonate salt.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A compound described herein may also be provided as a prodrug, which is a functional derivative of a compound, for example, of Formula I and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

The compounds described herein can be prepared, isolated, or obtained by any method known to one of ordinary skill in the art, for example, by following the procedures described in U.S. Pat. No. 10,376,511.

### Pharmaceutical Compositions

In one embodiment, provided herein is a pharmaceutical composition, comprising a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

A pharmaceutical composition provided herein can be formulated in various dosage forms, including, but not limited to, dosage forms for oral, parenteral, and topical administration. The pharmaceutical composition can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art. See, e.g., Remington: The Science and Practice of Pharmacy, supra; Modified-Release Drug Delivery Technology, 2nd ed.; Rathbone et al., Eds.; Drugs and the Pharmaceutical Sciences 184; CRC Press: Boca Raton, Fla., 2008.

In one embodiment, a pharmaceutical composition provided herein is formulated in a dosage form for oral administration. In another embodiment, a pharmaceutical composition provided herein is formulated in a dosage form for parenteral administration. In yet another embodiment, a pharmaceutical composition provided herein is formulated in a dosage form for intravenous administration. In yet another embodiment, a pharmaceutical composition provided herein is formulated in a dosage form for intramuscular administration. In yet another embodiment, a pharmaceutical composition provided herein is formulated in a dosage form for subcutaneous administration. In still another embodiment, a pharmaceutical composition provided herein is formulated in a dosage form for topical administration.

A pharmaceutical composition provided herein can be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient(s) (e.g., a compound provided herein) sufficient to produce the desired therapeutic effect, in association with the required pharmaceutical excipient(s). Examples of a unit-dosage form include, but are not limited to, an ampoule, syringe, and individually packaged tablet and capsule. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial, bottle of tablets or capsules, or bottle of pints or gallons.

A pharmaceutical composition provided herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

In one embodiment, a pharmaceutical composition provided herein comprises a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and sugar beads, talc, and povidone.

In another embodiment, a pharmaceutical composition provided herein comprises compound A22, or a tautomer, a mixture of two or more tautomers, or an isotopic variant thereof; or a pharmaceutically acceptable salt, solvate, hydrate, or prodrug thereof; and sugar beads, talc, and povidone.

In yet another embodiment, a pharmaceutical composition provided herein comprises compound A22 or a pharmaceutically acceptable salt; and sugar beads, talc, and povidone. In one embodiment, the pharmaceutical composition is formulated as a capsule.

In certain embodiments, a pharmaceutical composition provided herein comprises compound A22 or a pharmaceutically acceptable salt in an amount ranging from about 0.1 to about 50, from about 0.2 to about 20, from about 0.5 to about 10, or from about 0.5 to about 5 mg per capsule. In certain embodiments, a pharmaceutical composition provided herein comprises compound A22 or a pharmaceutically acceptable salt in an amount ranging from about 0.1 to about 50 mg per capsule. In certain embodiments, a pharmaceutical composition provided herein comprises compound A22 or a pharmaceutically acceptable salt in an amount ranging from about 0.2 to about 20 mg per capsule. In certain embodiments, a pharmaceutical composition provided herein comprises compound A22 or a pharmaceutically acceptable salt in an amount ranging from about 0.5 to about 10 mg per capsule. In certain embodiments, a pharmaceutical composition provided herein comprises compound A22 or a pharmaceutically acceptable salt in an amount ranging from about 0.5 to about 5 mg per capsule.

In certain embodiments, a pharmaceutical composition provided herein comprises compound A22 or a pharmaceutically acceptable salt in an amount of about 0.5, about 0.6, about 0.7, about 0.8, about 0.9, about 1, about 1.2, about 1.4, about 1.6, about 1.8, about 2, about 2.5, about 3, about 3.5, about 4, about 4.5, about 5, about 6, about 8, about 10, about 12, about 15, about 17, or about 20 mg per capsule. In certain embodiments, a pharmaceutical composition provided herein comprises compound A22 or a pharmaceutically acceptable salt in an amount of about 0.5, about 1, or about 2 mg per capsule.

In yet another embodiment, a pharmaceutical composition provided herein comprises a p-toluenesulfonate salt of compound A22; and sugar beads, talc, and povidone. In one embodiment, the pharmaceutical composition is formulated as a capsule.

In certain embodiments, a pharmaceutical composition provided herein comprises a p-toluenesulfonate salt of compound A22 in an amount ranging from about 0.1 to about 50, from about 0.2 to about 20, from about 0.5 to about 10, or from about 0.5 to about 5 mg per capsule. In certain embodiments, a pharmaceutical composition provided herein comprises a p-toluenesulfonate salt of compound A22 in an amount ranging from about 0.1 to about 50 mg per capsule. In certain embodiments, a pharmaceutical composition provided herein comprises a p-toluenesulfonate salt of compound A22 in an amount ranging from about 0.2 to about 20 mg per capsule. In certain embodiments, a pharmaceutical composition provided herein comprises a p-toluenesulfonate salt of compound A22 in an amount ranging from about 0.5 to about 10 mg per capsule. In certain embodiments, a pharmaceutical composition provided herein comprises a p-toluenesulfonate salt of compound A22 in an amount ranging from about 0.5 to about 5 mg per capsule.

In certain embodiments, a pharmaceutical composition provided herein comprises a p-toluenesulfonate salt of compound A22 in an amount of about 0.5, about 0.6, about 0.7, about 0.8, about 0.9, about 1, about 1.2, about 1.4, about 1.6, about 1.8, about 2, about 2.5, about 3, about 3.5, about 4, about 4.5, about 5, about 6, about 8, about 10, about 12, about 15, about 17, or about 20 mg per capsule. In certain embodiments, a pharmaceutical composition provided herein comprises a p-toluenesulfonate salt of compound A22 in an amount of about 0.5, about 1, or about 2 mg per capsule.

In still another embodiment, a pharmaceutical composition provided herein comprises a di-p-toluenesulfonate salt of compound A22; and sugar beads, talc, and povidone. In one embodiment, the pharmaceutical composition is formulated as a capsule.

In certain embodiments, a pharmaceutical composition provided herein comprises a di-p-toluenesulfonate salt of compound A22 in an amount ranging from about 0.1 to about 50, from about 0.2 to about 20, from about 0.5 to about 10, or from about 0.5 to about 5 mg per capsule. In certain embodiments, a pharmaceutical composition provided herein comprises a di-p-toluenesulfonate salt of compound A22 in an amount ranging from about 0.1 to about 50 mg per capsule. In certain embodiments, a pharmaceutical composition provided herein comprises a di-p-toluenesulfonate salt of compound A22 in an amount ranging from about 0.2 to about 20 mg per capsule. In certain embodiments, a pharmaceutical composition provided herein comprises a di-p-toluenesulfonate salt of compound A22 in an amount ranging from about 0.5 to about 10 mg per capsule. In certain embodiments, a pharmaceutical composition provided herein comprises a di-p-toluenesulfonate salt of compound A22 in an amount ranging from about 0.5 to about 5 mg per capsule.

In certain embodiments, a pharmaceutical composition provided herein comprises a di-p-toluenesulfonate salt of compound A22 in an amount of about 0.5, about 0.6, about 0.7, about 0.8, about 0.9, about 1, about 1.2, about 1.4, about 1.6, about 1.8, about 2, about 2.5, about 3, about 3.5, about 4, about 4.5, about 5, about 6, about 8, about 10, about 12, about 15, about 17, or about 20 mg per capsule. In certain embodiments, a pharmaceutical composition provided herein comprises a di-p-toluenesulfonate salt of compound A22 in an amount of about 0.5, about 1, or about 2 mg per capsule.

In certain embodiments, a pharmaceutical composition provided herein is formulated as an immediate-release capsule with a size of, e.g., size 1.

Methods of Treatment

In one embodiment, provided herein is method of treating relapsed or refractory acute myeloid leukemia (AML) or high-risk myelodysplastic syndrome (MDS), comprising administering to a subject in need thereof a therapeutically effective amount of a compound described herein.

In certain embodiments, a method provided herein is for treating relapsed or refractory AML. In certain embodiments, a method provided herein is for treating relapsed AML. In certain embodiments, a method provided herein is for treating refractory AML. In certain embodiments, a method provided herein is for treating del(5q) AML. In certain embodiments, the AML subject has a cytogenetic abnormality. In certain embodiments, the AML subject bears del(5q). In certain embodiments, the AML is drug-resistant.

In certain embodiments, the AML is drug-resistant. In certain embodiments, the AML is resistant to arsenic trioxide, cyclophosphamide, cytarabine, daunorubicin, dexamethasone, doxorubicin, enasidenib, gemtuzumab ozogamicin, gilteritinib, glasdegib, idamycin, idarubicin, ivosidenib, midostaurin, mitoxantrone, thioguanine, venetoclax, and/or vincristine.

In certain embodiments, a method provided herein is for treating high-risk MDS. In certain embodiments, a method provided herein is for treating relapsed or refractory MDS. In certain embodiments, a method provided herein is for treating relapsed MDS. In certain embodiments, a method provided herein is for treating refractory MDS. In certain embodiments, a method provided herein is for treating relapsed or refractory high-risk MDS. In certain embodiments, a method provided herein is for treating relapsed high-risk MDS. In certain embodiments, a method provided herein is for treating refractory high-risk MDS. In certain embodiments, a method provided herein is for treating del(5q) MDS. In certain embodiments, the MDS subject has a cytogenetic abnormality. In certain embodiments, the MDS subject bears del(5q).

In certain embodiments, the MDS is drug-resistant. In certain embodiments, the MDS is resistant to azacitidine, decitabine, and/or lenalidomide.

In certain embodiments, the subject has failed a prior therapy. In other embodiments, the subject has failed more than one prior therapy.

In certain embodiments, the subject is a mammal. In certain embodiments, the subject is a human.

A method provided herein encompasses treating a subject regardless of patient's age, although some diseases are more common in certain age groups.

In certain embodiments, the therapeutically effective amount of a compound described herein, e.g., compound A22, is ranging from about 0.001 to about 10 mg/kg per day, from about 0.002 to about 5 mg/kg per day, from about 0.005 to about 2 mg/kg per day, from about 0.01 to about 1 mg/kg per day, or from about 0.01 to about 0.5 mg/kg per day. In one embodiment, the therapeutically effective amount of a compound described herein, e.g., compound A22, is ranging from about 0.001 to about 10 mg/kg per day. In another embodiment, the therapeutically effective amount of a compound described herein, e.g., compound A22, is ranging from about 0.002 to about 5 mg/kg per day. In yet another embodiment, the therapeutically effective amount of a compound described herein, e.g., compound A22, is ranging from about 0.005 to about 2 mg/kg per day. In yet another embodiment, the therapeutically effective amount of a compound described herein, e.g., compound A22, is ranging from about 0.01 to about 1 mg/kg per day. In yet another embodiment, the therapeutically effective amount of a compound described herein, e.g., compound A22, is ranging from about 0.01 to about 0.5 mg/kg per day. In still another embodiment, the therapeutically effective amount of a compound described herein, e.g., compound A22, is about 0.01, about 0.02, about 0.03, about 0.05, about 0.08, about 0.1, about 0.12, about 0.15, about 0.17, about 0.2, or about 0.25 mg/kg per day.

In certain embodiments, the therapeutically effective amount of a compound described herein, e.g., compound A22, is ranging from about 0.1 to about 200 mg per day, from about 0.2 to about 100 mg per day, from about 0.5 to about 50 mg per day, or from about 1 mg every other day to about 20 mg per day. In one embodiment, the therapeutically effective amount of a compound described herein, e.g., compound A22, is ranging from about 0.1 to about 200 mg per day. In another embodiment, the therapeutically effective amount of a compound described herein, e.g., compound A22, is ranging from about 0.2 to about 100 mg per day. In yet another embodiment, the therapeutically effective amount of a compound described herein, e.g., compound A22, is ranging from about 0.5 to about 50 mg per day. In yet another embodiment, the therapeutically effective amount of a compound described herein, e.g., compound A22, is ranging from about 1 to about 20 mg per day. In yet another embodiment, the therapeutically effective amount of a compound described herein, e.g., compound A22, is about 1, about 2, about 3, about 5, about 8, about 10, about 11, about 14, about 15, about 17, about 20, or about 25 mg per day.

In certain embodiments, the therapeutically effective amount of a compound described herein, e.g., compound A22, is ranging from about 1 to 500, from about 2 to 250, from about 5 to about 100, or from about 10 to about 50 mg per week. In one embodiment, the therapeutically effective amount of a compound described herein, e.g., compound A22, is ranging from about 1 to 500 mg per week. In another embodiment, the therapeutically effective amount of a compound described herein, e.g., compound A22, is ranging from about 2 to 250 mg per week. In yet another embodiment, the therapeutically effective amount of a compound described herein, e.g., compound A22, is ranging from about 5 to about 100 per week. In yet another embodiment, the therapeutically effective amount of a compound described herein, e.g., compound A22, is ranging from about 10 to about 50 mg per week. In still another embodiment, the therapeutically effective amount of a compound described herein, e.g., compound A22, is about 5, about 10, about 15, about 20, about 25, about 30, about 35, about 40, about 50, or about 60 mg per week.

In certain embodiments, the compound is administered at a dose of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 22 mg, about 24 mg, about 25 mg, about 28 mg, about 30 mg, about 33 mg, about 35 mg, about 38 mg, about 40 mg, about 42 mg, about 45 mg, about 48 mg, about 51 mg, about 52 mg, about 55 mg, about 58 mg, about 60 mg, about 62 mg, about 65 mg, about 68 mg, about 70 mg, about 72 mg, about 75 mg, about 78 mg, or about 80 mg per week. In certain embodiments, the compound is administered for 1 day, 2 days, 3 days, 4 days, 5 days, 6 days, or 7 days per week.

It is understood that the administered dose of a compound described herein can also be expressed in units other than mg/kg every other day. For example, doses for parenteral administration can be expressed as mg/m² per day. One of ordinary skill in the art would readily know how to convert doses from mg/kg per day to mg/m² per day to given either the height or weight of a subject or both. For example, a dose of 1 mg/m² per day for a 65 kg human is approximately equal to 58 mg/kg per day.

Depending on the disease to be treated and the subject's condition, a compound described herein may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration.

In one embodiment, a compound described herein, e.g., compound A22, is administered orally. In another embodiment, a compound described herein, e.g., compound A22, is administered parenterally. In yet another embodiment, a compound described herein, e.g., compound A22, is administered intravenously. In yet another embodiment, a compound described herein, e.g., compound A22, is administered intramuscularly. In yet another embodiment, a compound described herein, e.g., compound A22, is administered subcutaneously. In still another embodiment, a compound described herein, e.g., compound A22, is administered topically.

A compound described herein, e.g., compound A22, can be delivered as a single dose such as, e.g., a single bolus injection, or oral tablets or pills; or over time such as, e.g., continuous infusion over time or divided bolus doses over time. A compound described herein, e.g., compound A22, can be administered repetitively if necessary, for example, until the subject experiences stable disease or regression, or until the subject experiences disease progression or unacceptable toxicity. Stable disease or lack thereof is determined by a method known in the art such as evaluation of subject's symptoms, physical examination, visualization of the cancer that has been imaged using X-ray, CAT, PET, or MRI scan and other commonly accepted evaluation modalities.

A compound described herein, e.g., compound A22, can be administered once daily (QD), or divided into multiple daily doses such as twice daily (BID), and three times daily (TID). In addition, the administration can be continuous, i.e., every day, or intermittently. The term “intermittent” or “intermittently” as used herein is intended to mean stopping and starting at either regular or irregular intervals. For example, intermittent administration of a compound described herein, e.g., compound A22, is administration for one to six days per week, administration in cycles (e.g., daily administration for two to eight consecutive weeks, then a rest period with no administration for up to one week), or administration on alternate days.

It will be understood, however, that the specific dose level and frequency of dosage for any particular subject can be varied and will depend upon a variety of factors including the activity of the specific compound employed, e.g., compound A22, the metabolic stability and length of action of the compound, the age, body weight, general health, sex, diet, mode and time of administration, rate of excretion, drug combination, the severity of the particular condition, and the host undergoing therapy.

In certain embodiments, a compound described herein, e.g., compound A22, is cyclically administered to a subject to be treated. Cycling therapy involves the administration of the compound for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

Consequently, in one embodiment, a compound described herein, e.g., compound A22, is administered for a cycle of about one week, about two weeks, about three weeks, about four weeks, about five weeks, about six weeks, about eight weeks, or about ten weeks, with a rest period of about 1 day to about four weeks. In one embodiment, a compound described herein, e.g., compound A22, is administered for a cycle of three weeks, four weeks, five weeks, or six weeks with a rest period of 1, 3, 5, 7, 9, 12, or 14. In certain embodiments, the rest period is 7 days. In certain embodiments, the rest period is 14 days. In certain embodiments, the rest period is a period that is sufficient for bone marrow recovery. The frequency, number, and length of dosing cycles can be increased or decreased.

In one embodiment, a compound described herein, e.g., compound A22, is administered for three weeks in a 28-day cycle with a 7-day rest period. In one embodiment, in a 28-day cycle with a 7-day rest period, a compound described herein, e.g., compound A22, is administered every day for five days of a week. In another embodiment, in a 28-day cycle with a 7-day rest period, a compound described herein, e.g., compound A22, is administered on Days 1, 2, 3, 4, 5, 8, 9, 10, 11, 12, 15, 16, 17, 18, and 19. In one embodiment, in a 28-day cycle with a 7-day rest period, a compound described herein, e.g., compound A22, is administered every day for three days of a week. In another embodiment, in a 28-day cycle with a 7-day rest period, a compound described herein, e.g., compound A22, is administered on Days 1, 3, 5, 8, 10, 12, 15, 17, and 19.

In certain embodiments, the subject is treated with a compound described herein, e.g., compound A22, from about 1 to about 50, from about 2 to about 20, from about 2 to 10, or from about 4 to about 8 cycles. In certain embodiments, the subject is treated with a compound described herein, e.g., compound A22, from about 1 to about 50 cycles. In certain embodiments, the subject is treated with a compound described herein, e.g., compound A22, from about 2 to about 20 cycles. In certain embodiments, the subject is treated with a compound described herein, e.g., compound A22, from about 2 to 10 cycles. In certain embodiments, the subject is treated with a compound described herein, e.g., compound A22, from about 4 to about 8 cycles.

In one embodiment, provided herein is a method of inhibiting the growth of a cell, comprising contacting the cell with an effective amount of a compound of Formula (I), e.g., compound A22, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In another embodiment, provided herein is a method of modulating the activity of CK1α in a cell, comprising contacting the cell with a compound of Formula (I), e.g., compound A22, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is a method of inducing apoptosis in a cell, comprising contacting the cell with a compound of Formula (I), e.g., compound A22, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the cell is a cancerous cell. In certain embodiments, the cell is an AML cell. In certain embodiments, the cell is a relapsed or refractory AML cell. In certain embodiments, the cell is a relapsed AML cell. In certain embodiments, the cell is a refractory AML cell. In certain embodiments, the cell is a del(5q) AML cell. In certain embodiments, the cell is a cell of AML having a cytogenetic abnormality. In certain embodiments, the cell is a drug-resistant AML cell.

In certain embodiments, the cell is an MDS cell. In certain embodiments, the cell is a relapsed or refractory MDS cell. In certain embodiments, the cell is a relapsed MDS cell. In certain embodiments, the cell is a refractory MDS cell. In certain embodiments, the cell is a del(5q) MDS cell. In certain embodiments, the cell is a cell of MDS having a cytogenetic abnormality. In certain embodiments, the cell is a drug-resistant MDS cell.

The disclosure will be further understood by the following non-limiting examples.

### EXAMPLES

As used herein, the symbols and conventions used in these processes, schemes, and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); mL (microliters); mM (millimolar); mM (micromolar); mmol (millimoles); h (hour or hours); and min (minutes).

### Example 1

### Open Label, Escalating Multiple Dose Study to Evaluate the Safety, Toxicity, and Pharmacokinetics of Compound A22 Capsules in Subjects with Relapsed or Refractory Acute Myeloid Leukemia or High-Risk Myelodysplastic Syndrome

This is a multicenter, open label, nonrandomized, sequential dose escalation/cohort expansion, multiple dose study, evaluating the safety, toxicity, and pharmacokinetics as well as efficacy of compound A22 capsules in subjects with relapsed or refractory AML or high-risk MDS. The study has two phases: Phases Ia and Ib.

In Phase Ia, compound A22 as capsules is administered orally to a maximum of 35 subjects to determine the dose limiting toxicities (DLTs) and MTD. Dosing in this phase consists of the first cycle of therapy of 28 days. Compound A22 starting dose for Cohort 1 is 1 mg for 5 days per week at a maximum weekly dose of 5 mg. Beginning with Cohort 2, doses are adjusted to 3 days per week (e.g., Monday, Wednesday, and Friday). Barring DLT, sequential dose escalation of compound A22 is up to a total of eight dose levels to a maximum of 20 mg at a maximum weekly dose of 60 mg. The numbers of subjects and actual doses administered are determined in response to DLTs using a Bayesian optimal interval (BOIN) design. There is one subject in each of the first two cohorts (increased to 3 subjects if a subject experiences Grade 2 toxicity that is not clearly attributable to underlying disease). There are at least 3 subjects per cohort starting with Cohort 3. In all cohorts with more than 1 subject, enrollment is staggered such that there are at least 7 days between the enrollment of the first subject on a given dose level and subsequent subjects. There is a minimum of 14 days for safety and PK evaluations between the completion of Cycle 1 for a given cohort and the initiation of dosing in Cycle 1 for the next cohort.

Toxicity severity is graded according to the National Cancer Institute Common Terminology Criteria for Adverse Events (NCI CTCAE) Version 5.0. For purposes of dose escalation, the totality of accrued safety information across all cycles completed at the time of DEC data review is taken into consideration. A DLT is defined as a severe or clinically significant AE or abnormal laboratory value (Grade 3 or greater, unless otherwise specified), unless it is clearly related to disease progression, intercurrent illness, preexisting condition, or concomitant medications.

In Phase Ib, once the MTD is determined (as the dose for which the isotonic estimate of the toxicity rate is closest to the target toxicity rate of 0.3), an additional 15 subjects are enrolled for supplementary experience with safety and efficacy and to determine the RP2D (which may or may not differ from the MTD). Dosing in this phase of the study consists of the first cycle of therapy of 28 days). The DEC reviews cumulative safety/PK data in subjects treated in Phase Ib for DLTs, with DEC reviews scheduled after every 5 subjects complete a cycle of compound A22. The toxicities included in the DLT definition are the same as in Phase Ia and the same elimination boundaries are applied. Phase Ib continues at the MTD or highest dose achieved in Phase Ia. Phase Ib is stopped, if, after enrollment of at least 5 subjects, >30% of subjects experience DLT(s) or a maximum of 15 subjects complete Cycle 1.

Subjects who complete one cycle of compound A22 in either Phase Ia or Phase Ib may be offered continued access to study drug for up to eight 28-day cycles. Dosing continue at the assigned dose or may be increased (not to exceed a level already tolerated by at least 1 subject if Phase Ia is ongoing or the MTD/RP2D if already established). The DEC continues to review accruing safety/PK data, inclusive of all cycles, for subjects who continue with treatment. Following completion of Phase 1b, the DEC meet every 6 months during the Continued Treatment Phase. Ad hoc meetings are convened if needed in response to safety concerns, including concerns based on DLTs observed in Phase Ia, Phase 1b, or the Continued Treatment Phase of the study.

If late occurring DLTs are identified (occurring at a rate >30% at the Phase Ib dose or if DLTs occur at a higher rate than observed in Phase Ia or Phase 1b), enrollment of subsequent subjects is placed on hold until all accrued data are reviewed by the DEC and a decision is made on whether to proceed. The Continued Treatment Phase is stopped in response to late-occurring DLTs that cannot be managed by supportive care, dose reduction, dose interruption, or less frequent administration.

Once treatment has completed, subjects are contacted by telephone every 3 months for survival status and anticancer therapy; the cause of death is documented. Individual subjects are considered to have completed the study 2 years after their last treatment or upon death, whichever occurs first.

Sample is not be based on a statistical power calculation. In Phases 1a/1b, the number of subjects per dose cohort is based on an accepted accelerated dose escalation design (BOIN). The sample size for Phase Ia is 35 subjects; additional subjects may be enrolled in the event that a given subject either does not receive study drug or discontinues early for reasons other than safety and is not evaluable for toxicity. For Phase 1b, up to an additional 15 subjects are enrolled for additional experience with safety and efficacy.

Eligible subjects for the study are the ones between 18 years or older with documented diagnosis of AML or MDS according to the World Health Organization (WHO) classification and, with respect to MDS, which is high risk (high risk or very high risk. Subjects must have refractory or relapsed disease. Additional inclusion criteria for the eligible subjects include (i) ECOG performance status of no greater than 2; (ii) a life expectancy no less than 6 weeks at study entry; and (iii) adequate organ function: serum creatinine ≤1.5×ULN and total bilirubin ≤1.5×ULN (higher levels are acceptable if these can be attributed to ineffective erythropoiesis, leukemia organ involvement (when other causes of hepatic toxicity have been ruled out), or Gilbert's syndrome); and aspartate aminotransferase (AST) and/or alanine transaminase (ALT)≤2×ULN, unless considered due to leukemic organ involvement (when other causes of hepatic toxicity have been ruled out).

The study excludes those with (i) diagnosis of acute promyelocytic leukemia; or (ii) white blood cell (WBC) count >20×109/L at screening (hydroxyurea may be used to bring the WBC count below the threshold; subjects may be retested within the 28-day screening period following treatment). The study also excludes those who have (i) cancer chemotherapy (other than hydroxyurea) within 2 weeks prior to the start of study drug (Cycle 1, Day 1); (ii) transplantation within the 3 months prior to screening; or (iii) treatment with systemic immunosuppressive medications including high-dose steroids (>20 mg prednisolone or equivalent per day), or calcineurin inhibitors (e.g., cyclosporine, tacrolimus) for at least 1 week prior to screening, and sirolimus, mycophenylate mofetil, azathioprine, or ruxolitinib for at least 2 weeks prior to screening.

Study drug in phases Ia and Ib is orally administered, immediate-release capsules of 0.5, 1.0, and 2.0 mg each. One 28-day cycle of treatment consists of 3 weeks of treatment, followed by 1 week with no study drug. On Day 1 of each applicable cycle, the study drug is dispensed in separate bottles for each capsule strength, including a sufficient amount to complete the treatment cycle (with the exception that the 0.5-mg and 1.0-mg strengths are not be dispensed together).

The eight dosing levels in Phase Ia are listed in Table 1. The numbers of subjects and doses administered may be altered in response to toxicity or tolerability. Dosing in Phase Ib and the continued treatment phase are determined on the basis of Phase Ia (up to a maximum of eight cycles). Treatment is discontinued in the event of confirmed progression, unacceptable toxicity or prolonged time required for recovery, withdrawal of consent, or if the Investigator determines removal from the study is in the subject's best interest.

Following provision of written informed consent, eligibility for participation is assessed during a screening period occurring up to 28 days before the start of study drug. A BM aspirate, BM biopsy, and peripheral blood (PB) sample are required at screening for confirmation of diagnosis of AML or MDS. These are tested for cytogenetics with karyotype and fluorescence in situ hybridization (FISH), mutational profiling with next generation sequencing or other technologies, and immunophenotype determination by multiparameter flow cytometry. Assessment of ECOG performance status is assessed at screening; to remain eligible for dosing the subject must continue to be ECOG ≤2 on admission to the hospital on Day −1. Serum pregnancy testing (β-hCG) for women of childbearing potential and serology is required at screening.

TABLE 1
Dosing Levels in Phase 1a

| | | | | | Recommended | | |
|---|---|---|---|---|---|---|---|
| | | | Maximum Weekly | | Number of | | |
| | Planned | Planned | Doses^{c} (mg) | | Capsules per | | |
| | Daily | Number | 5 | 3 | Strength (mg) | | |
| | Dose^{b} | of | Days/ | Days/ | Daily Dose | | |
| Cohort^{a} | (mg) | Subjects | Week | Week | 0.5^{d} | 1.0 | 2.0 |
| 1 | 1 | 1^{e,f} | 5 | — | 0 | 1 | 0 |
| 2 | 3 | 1^{e,f} | — | 9 | 0 | 1 | 1 |
| 3 | 5 | 3^{f} | | 15 | 0 | 1 | 2 |
| 4 | 8 | At least 3 | | 24 | 0 | 0 | 4 |
| 5 | 11 | per Cohort^{f} | | 33 | 0 | 1 | 5 |
| 6 | 14 | | | 42 | 0 | 0 | 7 |
| 7 | 17 | | | 51 | 0 | 1 | 8 |
| 8 | 20 | | | 60 | 0 | 0 | 10 |

^{a}If the first subject dosed in Cohort 1 (1 mg/day) does not have a DLT during Cycle 1, the next subject is enrolled at the next dose level (Cohort 2 [3 mg/day]) and so on for the rest of the planned dose levels.
^{b}De-escalation in dose, if required in response to DLT, is of the protocol for the first four cohorts or, for subsequent cohorts, by 50% or to a mid-dose between the current dose and the previous lower dose with DLT less than 0.3.
^{c}Dosing interval may be reduced in response to toxicity or undue PK accumulation (either for a given subject or for a cohort).
^{d}Available for dose modification (de-escalation or intermediate dose) that deviates from the planned treatment groups.
^{e}Cohort size(s) is to 3 subjects if grade 2 adverse event(s) occur that are not clearly attributable to the underlying disease.
^{f}Enrollment is staggered such that there are at least 7 days between the enrollment of the first subject on a given dose level and subsequent subjects.

Subjects are admitted to the hospital on Day −1 through the first 5 days of Cycle 1, at a minimum (and for the first 5 days of any subsequent dose escalation, if applicable) for TLS prophylaxis and monitoring. Admission begins at least 24 hours prior to the first study drug dosing day (Day −1) and lasts at least until Day 5.

During Cycle 1, admission for the first 5 days also allows for PK blood sampling; subjects returns to the clinic for PK sampling on Days 6, 7, and 8, thus, subjects are offered the option to stay in the hospital until completion of Day 8 assessments. During the balance of Cycle 1, subjects are assessed twice a week in Weeks 2 and 3 and once in Week 4. During Cycles 2 through 6, scheduled assessments occur every other week. After Cycle 6, i.e., Cycles 7 and 8, scheduled assessments occur monthly. Safety, toxicity, PK, and efficacy assessments are performed at designated visits throughout the study. Unscheduled visits occur as needed in response to safety concerns. In the event the dose (either the dose given at one time or the cumulative weekly dose) is increased for a given subject, monitoring resumes as for Cycle 1 as described above.

PB and BM samples are collected throughout the study consistent with the clinical management of subjects with relapsed or refractory AML and HR-MDS. With the subject's initial signing of the ICF for participation in this study, extra samples of PB and BM aspirates are collected and stored for possible evaluation of potential exploratory pharmacodynamic (PD) markers, either in response to safety or efficacy signals at a given dose level. BM biopsies collected previously for assessment of disease progression may also be stored for these exploratory analyses. Similarly, leukapheresis samples may be saved from subjects who undergo this procedure to reduce WBC counts. In addition, based on emerging data and science, stored samples may be further analyzed for exploratory endpoints in the future and is described as such in the study ICF.

The End of Treatment (EOT) Visit is conducted within 14 to 28 days after the last dose of study drug is administered in a given living subject, regardless of the reason for discontinuation. Adverse events ≥Grade 2 ongoing at the EOT Visit are followed until the event resolves to ≤Grade 1, stabilizes, subjects start alternate therapy, returns to a status that is clinically acceptable in the judgment of Investigator, is lost to follow-up, or terminates with the subject death. After discontinuation of compound A22, all surviving subjects are contacted by telephone once every 3 months thereafter for up to 2 years or until death for assessment of survival status and bone marrow transplant (BMT) conditioning or other new antineoplastic therapies since discontinuation of study drug. Subjects with continued response are followed until this study is closed.

Response is evaluated at the end of each cycle based on BM biopsy and aspirate and PB. Serial samples are collected for response assessments every month and at the time of suspected response or progression. For responding subjects, PB, BM aspirate, and biopsies are collected every other month or at suspected time of progression.

The European LeukemiaNet (ELN) panel 2017 guidelines are used to define response for AML. The IWG Criteria is used to define response for MDS. These include disease-specific definitions of response (listed below), disease-free survival, and overall survival.

Response includes (i) CR, complete remission with incomplete blood count recovery (CRi), morphologic leukemia free state (MLFS), and partial remission (PR) for AML subjects; and (ii) CR, PR, and bone marrow CR for HR MDS subjects.

At each dose level in Phase Ia, blood samples are collected from each subject for measurement of plasma concentrations of compound A22 (and its demethylated metabolite, if applicable) during the first and third weeks of Cycle 1 as follows: (i) Days 1, 3, and 5 within 15 minutes before dosing (predose at Hour 0) and postdose at 1, 2, 3, 5, 8, and 12 hours; (ii) Days 2, 4, and 6 (corresponding to 24 hours after the most recent dose; to be collected predose if a dosing day); (iii) Day 7 (corresponding to 48 hours after the most recent dose); (iv) predose on Day 8 (corresponding to 72 hours after the most recent dose); and Day 15 predose and postdose at 1, 2, 3, 5, 8, and 12 hours.

Standard PK parameter values are calculated, including maximum observed plasma concentration (Cₘₐₓ), observed time of peak concentration (Tₘₐₓ), overall exposure (area under the plasma concentration curve, AUC), and elimination half-life. In addition, in response to a DLT or any significant safety concern, PK blood samples are collected from the affected subject in order to measure levels of compound A22.

Extra PB and BM aspirate samples at dose levels that may be associated with efficacy and/or selected toxicity may be collected and stored for analysis for possible exploratory association with response or biomarker analyses, including but not limited to, cytogenetic testing, gene sequencing, leukemia relevant protein expression by flow/mass cytometry, and gene expression profiling. BM biopsies collected previously for assessment of disease progression may also be stored for these exploratory analyses.

Cytogenetics and mutation panel may include: (i) gene expression levels of target super-enhancer (SE) genes (i.e., Mcl1, MYC, MYB, and MDM2) by digital droplet polymerase chain reaction (PCR); (ii) MCL1, MYC, MDM2, p53 protein expression levels; (iii) gene mutation analysis by next generation sequencing; (iv) ex vivo response of leukemic cells to compound A22; (v) frequency of lymphocyte subsets and levels of immunoglobulins in PB; (vi) immune competence status (e.g., Mantoux test or interferon gamma (IFN-γ) staining); and (vii) chromosomal translocation (t) by karyotype and FISH.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A method of treating relapsed or refractory acute myeloid leukemia (AML) or high-risk myelodysplastic syndrome (MDS), comprising administering to a subject in need thereof a therapeutically effective amount of a compound of Formula (I): 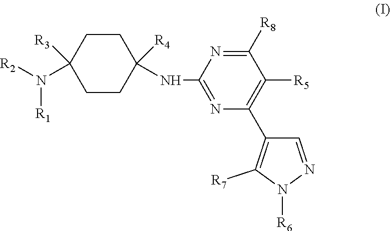
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
R₁ and R₂ are each independently (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; or
R₁ and R₂ together with the nitrogen atom to which they are attached form heteroaryl or heterocyclyl;
R₃ and R₄ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; or
R₁ or R₂ together with R₃ and the carbon and nitrogen atom to which they are attached form heterocyclyl;
R₅, R₇, and R₈ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R₆ is hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
wherein each alkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};
wherein each Q^{a} is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The method of claim 1, wherein R₁ is hydrogen.

3. The method of claim 1 or 2, wherein R₂ is hydrogen.

4. The method of any one of claims 1 to 3, wherein R₃ is hydrogen.

5. The method of any one of claims 1 to 4, wherein R₄ is hydrogen.

6. The method of any one of claims 1 to 5, wherein R₅ is hydrogen, deuterium, cyano, halo, nitro, or C₁₋₆ alkyl optionally substituted with one or more substituents Q.

7. The method of claim 6, wherein R₅ is halo.

8. The method of claim 6 or 7, wherein R₅ is Cl.

9. The method of any one of claims 1 to 8, wherein R₆ is hydrogen or C₁₋₆ alkyl optionally substituted with one or more substituents Q.

10. The method of claim 9, wherein R₆ is hydrogen.

11. The method of claim 9, wherein R₆ is methyl.

12. The method of any one of claims 1 to 11, wherein R₇ is C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; each of which is optionally substituents with one or more substituents Q.

13. The method of claim 12, wherein R₇ is C₁₋₆ alkyl optionally substituents with one or more substituents Q.

14. The method of claim 12, wherein R₇ is C₁₋₆ alkyl substituted with C₃₋₁₀ cycloalkyl, wherein the alkyl and cycloalkyl are each optionally substituents with one or more substituents Q^{a}.

15. The method of claim 12, wherein R₇ is cyclopropylmethyl, optionally substituents with one or more substituents Q.

16. The method of any one of claims 1 to 15, wherein R₈ is hydrogen, deuterium, cyano, halo, nitro, or C₁₋₆ alkyl optionally substituents with one or more substituents Q.

17. The method of claim 16, wherein R₈ is hydrogen.

18. The method of claim 1, wherein the compound is: 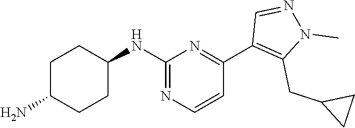 [SMILES: Cn1ncc(-c2ccnc(N[C@H]3CC[C@H](N)CC3)n2)c1CC1CC1]
(1r,4r)-N¹-(4-(5-(cyclopropylmethyl)-1-methyl-1H-pyrazol-4-yl)pyrimidin-2-yl)cyclohexane-1,4-diamine A1; 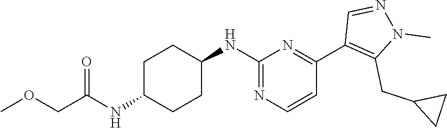 [SMILES: COCC(=O)N[C@H]1CC[C@H](Nc2nccc(-c3cnn(C)c3CC3CC3)n2)CC1]
N-((1r,4r)-4-((4-(5-(cyclopropylmethyl)-1-methyl-1H-pyrazol-4-yl)pyrimidin-2-yl)amino)cyclohexyl)-2-methoxyacetamide A2; 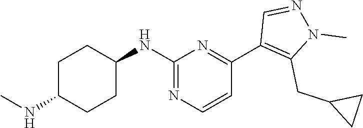 [SMILES: CN[C@H]1CC[C@H](Nc2nccc(-c3cnn(C)c3CC3CC3)n2)CC1]
(1r,4r)-N-(4-(5-(cyclopropylmethyl)-1-methyl-1H-pyrazol-4-yl)pyrimidin-2-yl)-N⁴-methylcyclohexane-1,4-diamine A3; 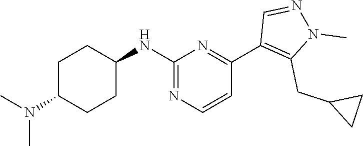 [SMILES: Cn1ncc(-c2ccnc(N[C@H]3CC[C@H](N(C)C)CC3)n2)c1CC1CC1]
(1r,4r)-N¹-(4-(5-(cyclopropylmethyl)-1-methyl-1H-pyrazol-4-yl)pyrimidin-2-yl)-N⁴,N⁴-dimethylcyclohexane-1,4-diamine A4; 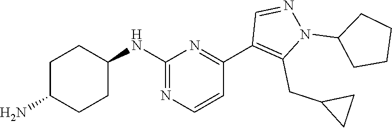 [SMILES: N[C@H]1CC[C@H](Nc2nccc(-c3cnn(C4CCCC4)c3CC3CC3)n2)CC1]
(1r,4r)-N¹-(4-(1-cyclopentyl-5-(cyclopropylmethyl)-1H-pyrazol-4-yl)pyrimidin-2-yl)cyclohexane-1,4-diamine A5; 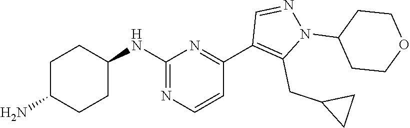 [SMILES: N[C@H]1CC[C@H](Nc2nccc(-c3cnn(C4CCOCC4)c3CC3CC3)n2)CC1]
(1r,4r)-N¹-(4-(5-(cyclopropylmethyl)-1-(tetrahydro-2H-pyran-4-yl)-1H-pyrazol-4-yl)pyrimidin-2-yl)cyclohexane-1,4-diamine A6; 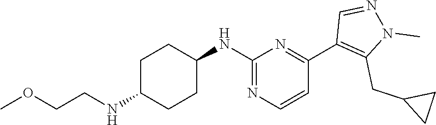 [SMILES: COCCN[C@H]1CC[C@H](Nc2nccc(-c3cnn(C)c3CC3CC3)n2)CC1]
(1r,4r)-N¹-4-(5-(cyclopropylmethyl)-1-methyl-1H-pyrazol-4-yl)pyrimidin-2-yl)-N⁴-(2-methoxyethyl)cyclohexane-1,4-diamine A7; 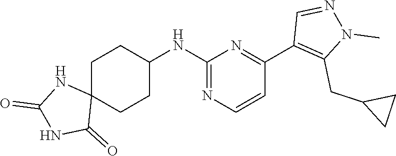 [SMILES: Cn1ncc(-c2ccnc(NC3CCC4(CC3)NC(=O)NC4=O)n2)c1CC1CC1]
8-((4-(5-(cyclopropylmethyl)-1-methyl-1H-pyrazol-4-yl)pyrimidin-2-yl)amino)-1,3-diazaspiro[4.5]decane-2,4-dione A8; 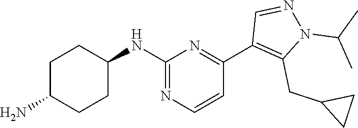 [SMILES: CC(C)n1ncc(-c2ccnc(N[C@H]3CC[C@H](N)CC3)n2)c1CC1CC1]
(1r,4r)-N¹-(4-(5-(cyclopropylmethyl)-1-isopropyl-1H-pyrazol-4-yl)pyrimidin-2-yl)cyclohexane-1,4-diamine A9; 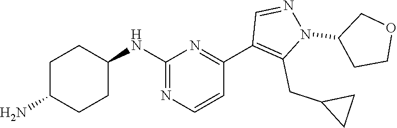
(1r,4S)—N¹-(4-(5-(cyclopropylmethyl)-1-((S)-tetrahydrofuran-3-yl)-1H-pyrazol-4-yl)pyrimidin-2-yl)cyclohexane-1,4-diamine A10; 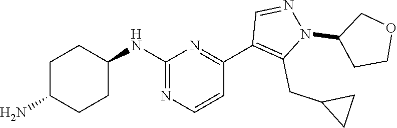
(1r,4S)—N¹-(4-(5-(cyclopropylmethyl)-1-((S)-tetrahydrofuran-3-yl)-1H-pyrazol-4-yl)pyrimidin-2-yl)cyclohexane-1,4-diamine A11; 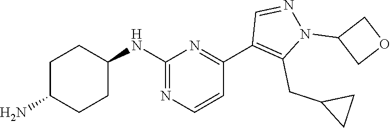 [SMILES: N[C@H]1CC[C@H](Nc2nccc(-c3cnn(C4COC4)c3CC3CC3)n2)CC1]
(1r,4r)-N¹-(4-(5-(cyclopropylmethyl)-1-(oxetan-3-yl)-1H-pyrazol-4-yl)pyrimidin-2-yl)cyclohexane-1,4-diamine A12; 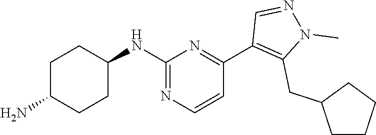 [SMILES: Cn1ncc(-c2ccnc(N[C@H]3CC[C@H](N)CC3)n2)c1CC1CCCC1]
(1r,4r)-N¹-(4-(5-(cyclopentylmethyl)-1-methyl-1H-pyrazol-4-yl)pyrimidin-2-yl)cyclohexane-1,4-diamine A13; 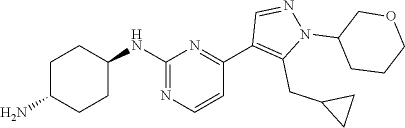 [SMILES: N[C@H]1CC[C@H](Nc2nccc(-c3cnn(C4CCCOC4)c3CC3CC3)n2)CC1]
(1r,4r)-N¹-(4-(5-(cyclopropylmethyl)-1-(tetrahydro-2H-pyran-3-yl)-1H-pyrazol-4-yl)pyrimidin-2-yl)cyclohexane-1,4-diamine A14; 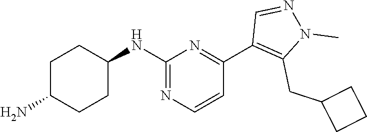 [SMILES: Cn1ncc(-c2ccnc(N[C@H]3CC[C@H](N)CC3)n2)c1CC1CCC1]
(1r,4r)-N¹-4-(5-(cyclobutylmethyl)-1-methyl-1H-pyrazol-4-yl)pyrimidin-2-yl)cyclohexane-1,4-diamine A15; 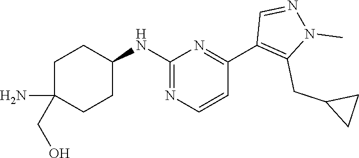
(1-amino-4-((4-(5-(cyclopropylmethyl)-1-methyl-1H-pyrazol-4-yl)pyrimidin-2-yl)amino)cyclohexyl)methanol A16; 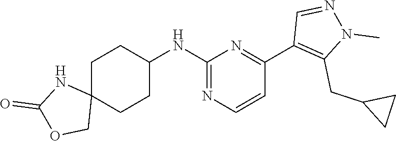 [SMILES: Cn1ncc(-c2ccnc(NC3CCC4(CC3)COC(=O)N4)n2)c1CC1CC1]
8-((4-(5-(cyclopropylmethyl)-1-methyl-1H-pyrazol-4-yl)pyrimidin-2-yl)amino)-3-oxa-1-azaspiro[4.5]decan-2-one A17; 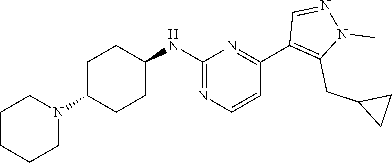 [SMILES: Cn1ncc(-c2ccnc(N[C@H]3CC[C@H](N4CCCCC4)CC3)n2)c1CC1CC1]
4-(5-(cyclopropylmethyl)-1-methyl-1H-pyrazol-4-yl)-N-((1r,4r)-4-(piperidin-1-yl)cyclohexyl)pyrimidin-2-amine A18; 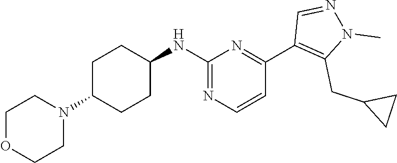 [SMILES: Cn1ncc(-c2ccnc(N[C@H]3CC[C@H](N4CCOCC4)CC3)n2)c1CC1CC1]
4-(5-(cyclopropylmethyl)-1-methyl-1H-pyrazol-4-yl)-N-((1r,4r)-4-morpholinocyclohexyl)-pyrimidin-2-amine A19; 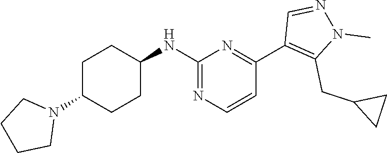 [SMILES: Cn1ncc(-c2ccnc(N[C@H]3CC[C@H](N4CCCC4)CC3)n2)c1CC1CC1]
4-(5-(cyclopropylmethyl)-1-methyl-1H-pyrazol-4-yl)-N-((1r,4r)-4-(pyrrolidin-1-yl)cyclohexyl)pyrimidin-2-amine A20; 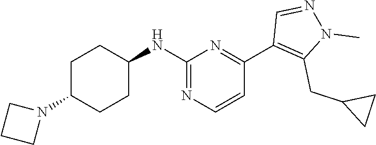 [SMILES: Cn1ncc(-c2ccnc(N[C@H]3CC[C@H](N4CCC4)CC3)n2)c1CC1CC1]
N-((1r,4r)-4-(azetidin-1-yl)cyclohexyl)-4-(5-(cyclopropylmethyl)-1-methyl-1H-pyrazol-4-yl)pyrimidin-2-amine A21; 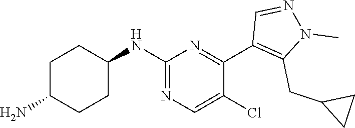 [SMILES: Cn1ncc(-c2nc(N[C@H]3CC[C@H](N)CC3)ncc2Cl)c1CC1CC1]
(1r,4r)-N¹-(5-chloro-4-(5-(cyclopropylmethyl)-1-methyl-1H-pyrazol-4-yl)pyrimidin-2-yl)cyclohexane-1,4-diamine A22; 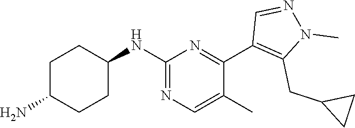 [SMILES: Cc1cnc(N[C@H]2CC[C@H](N)CC2)nc1-c1cnn(C)c1CC1CC1]
(1r,4r)-N¹-(4-(5-(cyclopropylmethyl)-1-methyl-1H-pyrazol-4-yl)-5-methylpyrimidin-2-yl)cyclohexane-1,4-diamine A23; 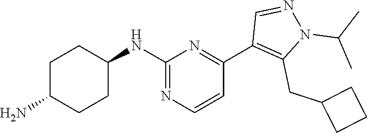 [SMILES: CC(C)n1ncc(-c2ccnc(N[C@H]3CC[C@H](N)CC3)n2)c1CC1CCC1]
(1r,4r)-N¹-(4-(5-(cyclobutylmethyl)-1-isopropyl-1H-pyrazol-4-yl)pyrimidin-2-yl)cyclohexane-1,4-diamine A24; 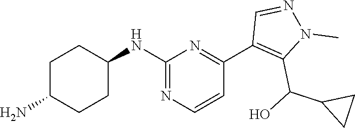 [SMILES: Cn1ncc(-c2ccnc(N[C@H]3CC[C@H](N)CC3)n2)c1C(O)C1CC1]
(4-(2-(((1r,4r)-4-aminocyclohexyl)amino)pyrimidin-4-yl)-1-methyl-1H-pyrazol-5-yl)(cyclopropyl)methanol A25; 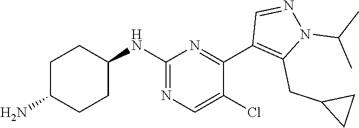 [SMILES: CC(C)n1ncc(-c2nc(N[C@H]3CC[C@H](N)CC3)ncc2Cl)c1CC1CC1]
(1r,4r)-N¹-(5-chloro-4-(5-(cyclopropylmethyl)-1-isopropyl-1H-pyrazol-4-yl)pyrimidin-2-yl)cyclohexane-1,4-diamine A26; 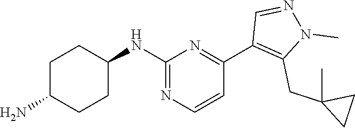 [SMILES: Cn1ncc(-c2ccnc(N[C@H]3CC[C@H](N)CC3)n2)c1CC1(C)CC1]
(1r,4r)-N¹-4-(1-methyl-5-((1-methylcyclopropyl)methyl)-1H-pyrazol-4-yl)pyrimidin-2-yl)cyclohexane-1,4-diamine A27; 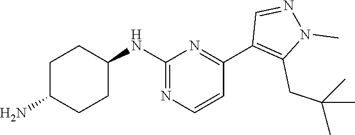 [SMILES: Cn1ncc(-c2ccnc(N[C@H]3CC[C@H](N)CC3)n2)c1CC(C)(C)C]
(1r,4r)-N¹-4-(1-methyl-5-neopentyl-1H-pyrazol-4-yl)pyrimidin-2-yl)cyclohexane-1,4-diamine A28; 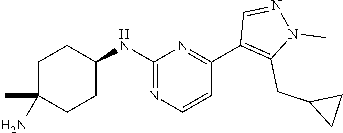 [SMILES: Cn1ncc(-c2ccnc(N[C@H]3CC[C@](C)(N)CC3)n2)c1CC1CC1]
(1r,4r)-N¹-(4-(5-(cyclopropylmethyl)-1-methyl-1H-pyrazol-4-yl)pyrimidin-2-yl)-4-methylcyclohexane-1,4-diamine A29; 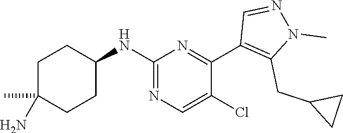 [SMILES: Cn1ncc(-c2nc(N[C@H]3CC[C@@](C)(N)CC3)ncc2Cl)c1CC1CC1]
is (1s,4s)-N¹-(5-chloro-4-(5-(cyclopropylmethyl)-1-methyl-1H-pyrazol-4-yl)pyrimidin-2-yl)-4-methylcyclohexane-1,4-diamine A30; 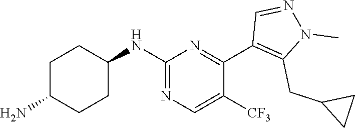 [SMILES: Cn1ncc(-c2nc(N[C@H]3CC[C@H](N)CC3)ncc2C(F)(F)F)c1CC1CC1]
(1r,4r)-N¹-(4-(5-(cyclopropylmethyl)-1-methyl-1H-pyrazol-4-yl)-5-(trifluoromethyl)-pyrimidin-2-yl)cyclohexane-1,4-diamine A31; 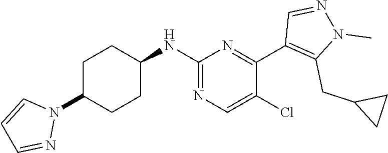 [SMILES: Cn1ncc(-c2nc(N[C@H]3CC[C@@H](n4cccn4)CC3)ncc2Cl)c1CC1CC1]
N-((1r,4r)-4-(1H-pyrazol-1-yl)cyclohexyl)-5-chloro-4-(5-(cyclopropylmethyl)-1-methyl-1H-pyrazol-4-yl)pyrimidin-2-amine A32; 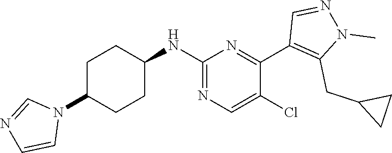 [SMILES: Cn1ncc(-c2nc(N[C@H]3CC[C@@H](n4ccnc4)CC3)ncc2Cl)c1CC1CC1]
N-((1r, 4r)-4-(1H-imidazol-1-yl)cyclohexyl)-5-chloro-4-(5-(cyclopropylmethyl)-1-methyl-1H-pyrazol-4-yl)pyrimidin-2-amine A33; 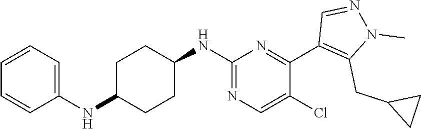 [SMILES: Cn1ncc(-c2nc(N[C@H]3CC[C@@H](Nc4ccccc4)CC3)ncc2Cl)c1CC1CC1]
(1r,4r)-N¹-(5-chloro-4-(5-(cyclopropylmethyl)-1-methyl-1H-pyrazol-4-yl)pyrimidin-2-yl)-N⁴-phenylcyclohexane-1,4-diamine A34; 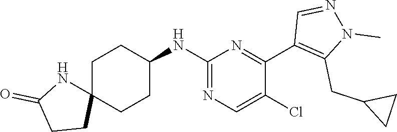
(5r,8r)-8-((5-chloro-4-(5-(cyclopropylmethyl)-1-methyl-1H-pyrazol-4-yl)pyrimidin-2-yl)amino)-1-azaspiro[4.5]decan-2-one A35; 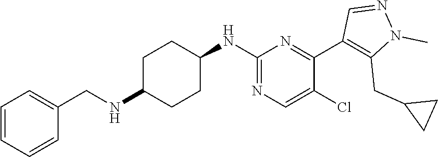
(1r,4r)-N¹-benzyl-N⁴-(5-chloro-4-(5-(cyclopropylmethyl)-1-methyl-1H-pyrazol-4-yl)pyrimidin-2-yl)cyclohexane-1,4-diamine A36; 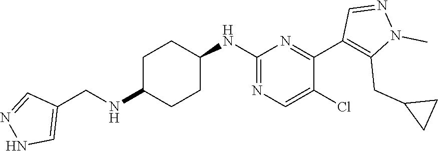
(1r,4r)-N¹-((1H-pyrazol-4-yl)methyl)-N⁴-(5-chloro-4-(5-(cyclopropylmethyl)-1-methyl-1H-pyrazol-4-yl)pyrimidin-2-yl)cyclohexane-1,4-diamine A37; 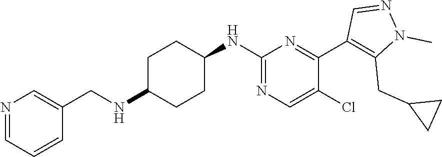
(1r,4r)-N¹-(5-chloro-4-(5-(cyclopropylmethyl)-1-methyl-1H-pyrazol-4-yl)pyrimidin-2-yl)-N⁴-(pyridin-3-ylmethyl)cyclohexane-1,4-diamine A38; 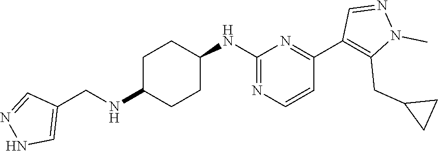
(1r,4r)-N¹-((1H-pyrazol-4-yl)methyl)-N⁴-(4-(5-(cyclopropylmethyl)-1-methyl-1H-pyrazol-4-yl)pyrimidin-2-yl)cyclohexane-1,4-diamine A39; 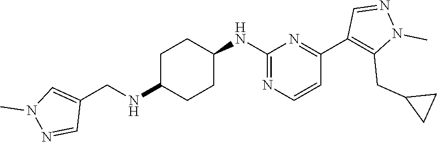
(1r,4r)-N¹-(4-(5-(cyclopropylmethyl)-1-methyl-1H-pyrazol-4-yl)pyrimidin-2-yl)-N⁴-((1-methyl-1H-pyrazol-4-yl)methyl)cyclohexane-1,4-diamine A40; 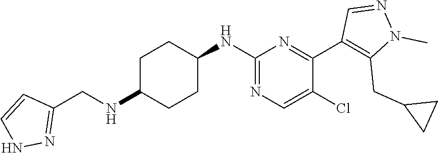
(1r,4r)-N¹-((1H-pyrazol-5-yl)methyl)-N⁴-(5-chloro-4-(5-(cyclopropylmethyl)-1-methyl-1H-pyrazol-4-yl)pyrimidin-2-yl)cyclohexane-1,4-diamine A41; 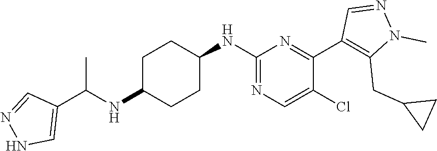
(1r,4r)-N¹-(1-(1H-pyrazol-4-yl)ethyl)-N⁴-(5-chloro-4-(5-(cyclopropylmethyl)-1-methyl-1H-pyrazol-4-yl)pyrimidin-2-yl)cyclohexane-1,4-diamine A42; 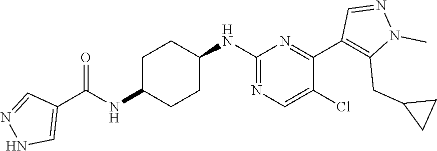
(1r,4r)-N¹-(5-chloro-4-(5-(cyclopropylmethyl)-1-methyl-1H-pyrazol-4-yl)pyrimidin-2-yl)-N⁴-phenylcyclohexane-1,4-diamine A43; 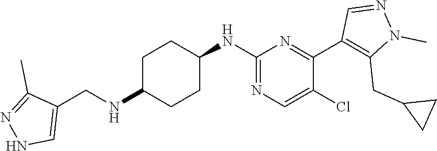
(1r,4r)-N¹-(5-chloro-4-(5-(cyclopropylmethyl)-1-methyl-1H-pyrazol-4-yl)pyrimidin-2-yl)-N⁴-((5-methyl-1H-pyrazol-4-yl)methyl)cyclohexane-1,4-diamine A44; 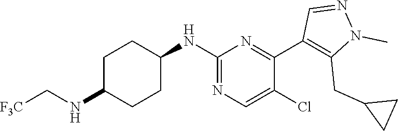
(1r,4r)-N¹-(5-chloro-4-(5-(cyclopropylmethyl)-1-methyl-1H-pyrazol-4-yl)pyrimidin-2-yl)-N⁴-(2,2,2-trifluoroethyl)cyclohexane-1,4-diamine A45; 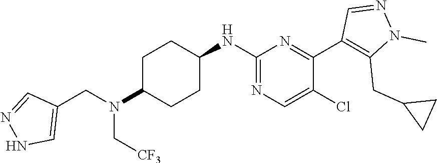
(1r,4r)-N¹-((1H-pyrazol-4-yl)methyl)-N⁴-(5-chloro-4-(5-(cyclopropylmethyl)-1-methyl-1H-pyrazol-4-yl)pyrimidin-2-yl)-N¹-(2,2,2-trifluoroethyl)cyclohexane-1,4-diamine A46; 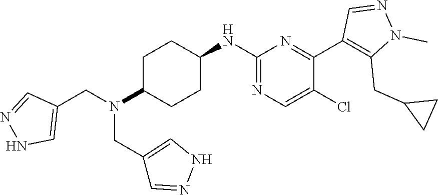
(1r,4r)-N′,N′-bis((1H-pyrazol-4-yl)methyl)-N⁴-(5-chloro-4-(5-(cyclopropylmethyl)-1-methyl-1H-pyrazol-4-yl)pyrimidin-2-yl)cyclohexane-1,4-diamine A47; 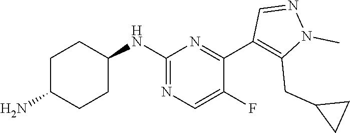 [SMILES: Cn1ncc(-c2nc(N[C@H]3CC[C@H](N)CC3)ncc2F)c1CC1CC1]
(1r,4r)-N′-(4-(5-(cyclopropylmethyl)-1-methyl-1H-pyrazol-4-yl)-5-fluoropyrimidin-2-yl)cyclohexane-1,4-diamine A48; 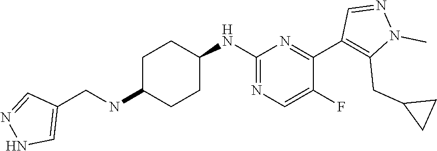
(1r,4r)-N¹-((1H-pyrazol-4-yl)methyl)-N⁴-(4-(5-(cyclopropylmethyl)-1-methyl-1H-pyrazol-4-yl)-5-fluoropyrimidin-2-yl)cyclohexane-1,4-diamine A49; or 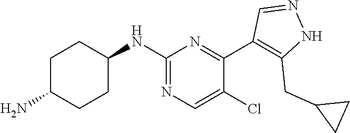 [SMILES: N[C@H]1CC[C@H](Nc2ncc(Cl)c(-c3cn[nH]c3CC3CC3)n2)CC1]
(1r,4r)-N¹-(5-chloro-4-(5-(cyclopropylmethyl)-1H-pyrazol-4-yl)pyrimidin-2-yl)cyclohexane-1,4-diamine A50;
or a tautomer, a mixture of two or more tautomers, or an isotopic variant thereof; or a pharmaceutically acceptable salt, solvate, hydrate, or prodrug thereof.

19. The method of claim 1, wherein the compound is (1r,4r)-N¹-(5-chloro-4-(5-(cyclopropylmethyl)-1-methyl-1H-pyrazol-4-yl)pyrimidin-2-yl)cyclohexane-1,4-diamine A22, or a tautomer, a mixture of two or more tautomers, or an isotopic variant thereof; or a pharmaceutically acceptable salt, solvate, hydrate, or prodrug thereof.

20. The method of claim 1, wherein the compound is (1r,4r)-N-(5-chloro-4-(5-(cyclopropylmethyl)-1H-pyrazol-4-yl)pyrimidin-2-yl)cyclohexane-1,4-diamine A50, or a tautomer, a mixture of two or more tautomers, or an isotopic variant thereof; or a pharmaceutically acceptable salt, solvate, hydrate, or prodrug thereof.

21. The method of any one of claims 1 to 20, wherein the method is for treating relapsed or refractory AML.

22. The method of claim 21, wherein the AML is relapsed.

23. The method of claim 21 or 22, wherein the AML is refractory.

24. The method of any one of claims 21 to 23, wherein the AML is del(5q) AML.

25. The method of any one of claims 21 to 24, wherein the AML is drug-resistant.

26. The method of any one of claims 1 to 20, wherein the method is for treating relapsed or refractory high-risk MDS.

27. The method of claim 26, wherein the MDS is relapsed.

28. The method of claim 26 or 27, wherein the MDS is refractory.

29. The method of any one of claims 26 to 28, wherein the MDS is del(5q) MDS.

30. The method of any one of claims 26 to 29, wherein the MDS is drug-resistant.

31. The method of any one of claims 1 to 30, wherein the subject has failed a prior therapy.

32. The method of any one of claims 1 to 31, wherein the subject is a human.

33. The method of any one of claims 1 to 32, wherein the compound is administered orally.

34. The method of any one of claims 1 to 33, wherein the compound is administered as a tablet or capsule.

35. The method of any one of claims 1 to 34, wherein the therapeutically effective amount is ranging from about 0.001 to about 10 mg/kg per day.

36. The method of any one of claims 1 to 35, wherein the therapeutically effective amount is ranging from about 0.1 to about 200 mg per day.

37. The method of any one of claims 1 to 36, wherein the therapeutically effective amount is about 1, about 2, about 3, about 5, about 8, about 10, about 11, about 14, about 15, about 17, about 20, or about 25 mg per day.

38. The method of any one of claims 1 to 37, wherein the compound is administered in a cycle.

39. The method of any one of claims 1 to 38, wherein the compound is administered in a 28-cycle.

40. The method of any one of claims 1 to 39, wherein the compound is administered for 1 day, 2 days, 3 days, 4 days, 5 days, 6 days, or 7 days per week.

41. The method of any one of claims 1 to 40, wherein the compound is administered for 3 days or 5 days per week.

42. The method of any one of claims 1 to 41, wherein the compound is administered on Days 1, 2, and 3 in a week.

43. The method of any one of claims 1 to 42, wherein the compound is administered on Days 1, 2, 3, 4, and 5 in a week.

44. The method of any one of claims 1 to 43, wherein the compound is administered in a 28-day cycle for 1 day, 2 days, 3 days, 4 days, 5 days, 6 days, or 7 days per week for 3 weeks, followed by 1 week of rest.

45. The method of any one of claims 1 to 44, wherein the compound is administered in a 28-day cycle for 5 days per week for 3 weeks followed by 1 week of rest.

46. The method of any one of claims 1 to 45, wherein the compound is administered in a 28-day cycle on Days 1, 2, 3, 4, and 5 per week for 3 weeks followed by 1 week of rest.

47. The method of any one of claims 1 to 44, wherein the compound is administered in a 28-day cycle for 3 days per week for 3 weeks followed by 1 week of rest.

48. The method of claim 47, wherein the compound is administered in a 28-day cycle on Days 1, 3, and 5 per week for 3 weeks followed by 1 week of rest.
